(19) United States
(12) Patent Application Publication
(10) Pub. No.: US 2024/0192223 A1
(43) Pub. Date: Jun. 13, 2024

(54) METHODS AND MATERIALS FOR IDENTIFYING AND TREATING MONOCLONAL AND OLIGOCLONAL GAMMOPATHIES

(71) Applicant: Mayo Foundation for Medical Education and Research, Rochester, MN (US)

(72) Inventors: David L. Murray, Rochester, MN (US); Anand Padmanabhan, Rochester, MN (US)

(21) Appl. No.: 18/285,691

(22) PCT Filed: Jun. 10, 2022
(86) PCT No.: PCT/US2022/032950

§ 371 Date: Oct. 5, 2023

Related U.S. Application Data
(60) Provisional application No. 63/209,217, filed on Jun. 10, 2021.

(51) Int. Cl. G01N 33/68

(52) U.S. Cl. CPC G01N 33/6854; G01N 2333/522; G01N 2800/226

(57) ABSTRACT

This document relates to methods and materials for assessing and/or treating mammals (e.g., humans) having a monoclonal or oligoclonal gammopathy of thrombotic/thrombocytopenia significance (MGTS). For example, the presence of a population of monoclonal antibodies having binding specificity for a platelet factor 4 (PF4) polypeptide in a sample obtained from a mammal (e.g., a human) can be used to identify the mammal as having MGTS. In some cases, materials and methods for treating a mammal (e.g., a human) identified as having MGTS are provided.

### CROSS-REFERENCE TO RELATED APPLICATIONS

This application claims the benefit of U.S. Patent Application Ser. No. 63/209,217, filed on Jun. 10, 2021. The disclosure of the prior application is considered part of (and is incorporated by reference in) the disclosure of this application.

### STATEMENT REGARDING FEDERAL FUNDING

This invention was made with government support under HL133479 awarded by the National Institutes of Health. The government has certain rights in the invention.

### BACKGROUND

### 1. Technical Field

This document relates to methods and materials for assessing and/or treating mammals (e.g., humans) having a monoclonal or oligoclonal gammopathy of thrombotic/thrombocytopenia significance (MGTS). For example, the presence of a population of monoclonal antibodies having binding specificity for a platelet factor 4 (PF4) polypeptide in a sample obtained from a mammal (e.g., a human) having one or more thrombotic and/or thrombocytopenia conditions can be used to identify the mammal as having MGTS. For example, the presence of a population of oligoclonal antibodies having binding specificity for a platelet factor 4 (PF4) polypeptide in a sample obtained from a mammal (e.g., a human) having one or more thrombotic and/or thrombocytopenic conditions can be used to identify the mammal as having MGTS. Also provided are materials and methods for treating mammals (e.g., a human) identified as having MGTS.

### 2. Background Information

Heparin-induced thrombocytopenia (HIT) is a severe adverse reaction to the commonly used anticoagulant heparin characterized by antibodies to PF4 polypeptides and PF4-polyanion complexes. In classical HIT, thrombocytopenia develops 1-2 weeks after heparin exposure and is complicated by thrombosis in 30-50% of patients. Spontaneous HIT refers to thrombocytopenia with or without thrombosis due to anti-PF4 antibodies that occurs in the absence of proximate heparin exposure and has been noted in multiple settings including but not limited to post-orthopedic surgery, after viral/bacterial prodromes, after vaccinations (e.g. after certain COVID-19 vaccinations) and other unknown triggers.

### SUMMARY

This document provides methods and materials for identifying and/or treating mammals (e.g., humans) having, or at risk of developing, MGTS. For example, this document provides methods and materials for identifying a mammal (e.g., a human) having one or more thrombotic and/or thrombocytopenia conditions as having, or as being at risk of developing, MGTS based, at least in part, on the presence of a population of monoclonal antibodies having binding specificity for a PF4 polypeptide in a sample obtained from the mammal. For example, this document provides methods and materials for identifying a mammal (e.g., a human) having one or more thrombotic and/or thrombocytopenic conditions as having, or as being at risk of developing, MGTS based, at least in part, on the presence of a population of oligoclonal antibodies having binding specificity for a PF4 polypeptide in a sample obtained from the mammal. This document also provides methods and materials for detecting a population of monoclonal antibodies having binding specificity for a PF4 polypeptide and/or a population of oligoclonal antibodies having binding specificity for a PF4 polypeptide in a sample obtained from a mammal (e.g., a human).

As described herein, mammals (e.g., humans) having HIT or spontaneous HIT can develop monoclonal or oligoclonal antibodies having binding specificity for a PF4 polypeptide (anti-PF4 antibodies) that causes MGTS. Also described herein are methods for detecting the presence of a population of monoclonal or oligoclonal antibodies having binding specificity for a PF4 polypeptide in a sample obtained from a mammal (e.g., a human). For example, a sample obtained from a mammal (e.g., a human) can be evaluated for clonality of anti-PF4 antibodies by coupling an anti-PF4 antibody enrichment with a mass spectrometry technique. Identifying mammals (e.g., humans) having one or more thrombotic and/or thrombocytopenia conditions having, or as being at risk of developing, MGTS can allow clinicians and patients to proceed with appropriate treatment options.

In general, one aspect of this document features methods for detecting a population of monoclonal or oligoclonal antibodies specific for a PF4 polypeptide in a sample obtained from a mammal. The methods can include, or consist essentially of, immunopurifying antibodies specific for a PF4 polypeptide from a sample obtained from a mammal; subjecting the immunopurified immunoglobulins to a mass spectrometry technique to obtain a mass spectrum of the sample; and identifying the presence of the population of monoclonal antibodies specific for the PF4 polypeptide based on a peak in the spectrum corresponding to the monoclonal or oligoclonal antibodies specific for the PF4 polypeptide. The immunopurifying can include contacting the sample with agarose beads comprising PF4 polypeptides. The mass spectrometry technique can include liquid chromatography. The mass spectrometry technique can be a quadrupole time-of-flight (TOF) mass spectrometry technique using electrospray ionization (ESI-Q-TOF mass spectrometry) or a matrix-assisted laser desorption/ionization (MALDI)-TOF mass spectrometry. The sample can be a serum sample or a plasma sample. The mammal can be a human. The method can include assessing the population of the monoclonal or oligoclonal antibodies specific for the PF4 polypeptide for platelet activating activity. The method can include assessing the population of the monoclonal or oligoclonal antibodies specific for the PF4 polypeptide for the ability to bind the PF4 polypeptide. The method can include assessing the population of the monoclonal or oligoclonal antibodies specific for the PF4 polypeptide for the ability to bind a complex comprising the PF4 polypeptide and a polyanion.

In another aspect, this document features methods for assessing a mammal having a thrombotic and/or thrombocytopenic condition. The methods can include, or consist essentially of, (a) detecting, in a sample from a mammal having a thrombotic and/or thrombocytopenia condition, a presence or absence of a population of monoclonal or oligoclonal antibodies having binding specificity for a PF4 polypeptide; (b) classifying the mammal as having MGTS if the presence of said population is detected; and (c) classifying the mammal as not having MGTS if the absence of the population level is detected. The thrombotic and/or thrombocytopenic condition can be heparin-induced thrombocytopenia (HIT), HIT with thrombosis, delayed-onset HIT, flush-related HIT, persistent HIT, HIT with disseminated intravascular coagulation, refractory HIT, spontaneous HIT with thrombosis, or spontaneous HIT without thrombosis. The detecting step can include a technique selected from the group consisting of a mass spectrometry technique, immunofixation electrophoresis (IFE), and serum protein electrophoresis (SPEP). The mass spectrometry technique can include liquid chromatography. The mass spectrometry technique can be ESI-Q-TOF mass spectrometry or MALDI-TOF mass spectrometry. The method can include, prior to the detecting, immunopurifying the sample for antibodies specific for the PF4 polypeptide. The immunopurifying can include contacting the sample with agarose beads comprising PF4 polypeptides. The sample can be a serum sample or a plasma sample. The mammal can be a human. When the presence of the presence of the population is detected, the method can include assessing the population of the monoclonal or oligoclonal antibodies specific for the PF4 polypeptide for platelet activating activity. When the presence of the presence of the population is detected, the method can include assessing the population of the monoclonal or oligoclonal antibodies specific for the PF4 polypeptide for the ability to bind the PF4 polypeptide. When the presence of the presence of the population is detected, the method can include assessing the monoclonal or oligoclonal antibodies specific for the PF4 polypeptide for the ability to bind a complex comprising the PF4 polypeptide and a polyanion.

In another aspect, this document features methods for treating a mammal having a thrombotic and/or thrombocytopenic condition. The methods can include, or consist essentially of, (a) detecting, in a sample obtained from a mammal having a thrombotic and/or thrombocytopenia condition, a population of monoclonal or oligoclonal antibodies having binding specificity for a PF4 polypeptide; and (b) administering a therapy effective to treat a monoclonal gammopathy to the mammal. The therapy can be a B-cell targeted therapy, a plasma cell targeted therapy, plasmapheresis, therapeutic plasma exchange, or administering intravenous immunoglobulin G. The detecting step can include a technique selected from the group consisting of a mass spectrometry technique, IFE, and SPEP. The mass spectrometry technique can include liquid chromatography. The mass spectrometry technique can be ESI-Q-TOF mass spectrometry or MALDI-TOF mass spectrometry. The method can include, prior to the detecting, immunopurifying the sample for antibodies specific for the PF4 polypeptide. The immunopurifying can include contacting the sample with agarose beads comprising PF4 polypeptides. The sample can be a serum sample or a plasma sample. The mammal can be a human. The method can include assessing the population of the monoclonal or oligoclonal antibodies specific for the PF4 polypeptide for platelet activating activity. The method can include assessing the population of the monoclonal or oligoclonal antibodies specific for the PF4 polypeptide for the ability to bind the PF4 polypeptide. The method can include assessing the population of the monoclonal or oligoclonal antibodies specific for the PF4 polypeptide for the ability to bind a complex comprising the PF4 polypeptide and a polyanion.

In another aspect, this document features methods for a mammal having a thrombotic and/or thrombocytopenia condition. The methods can include, or consist essentially of, (a) detecting, in a sample obtained from a mammal having a thrombotic and/or thrombocytopenia condition, an absence of a population of monoclonal or oligoclonal antibodies having binding specificity for a PF4 polypeptide; and (b) administering a therapy effective to treat thrombosis to said mammal. The therapy can be an anticoagulant, administering a thrombolytic, therapeutic plasma exchange, or administering intravenous immunoglobulin G. The detecting step can include a technique selected from the group consisting of a mass spectrometry technique, IFE, and SPEP. The mass spectrometry technique can include liquid chromatography. The mass spectrometry technique can be ESI-Q-TOF mass spectrometry or MALDI-TOF mass spectrometry. The method can include, prior to the detecting, immunopurifying the sample for antibodies specific for the PF4 polypeptide. The immunopurifying can include contacting the sample with agarose beads comprising PF4 polypeptides. The sample can be a serum sample or a plasma sample. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Anti-PF4 antibody enrichment. Heparin Sepharose® beads were treated with PF4 and then incubated with patient sample. Antibody bound to beads were eluted with high salt, dialyzed, and used for mass spectrometry studies.

FIG. 2. Schematic representation of the experimental workflow to characterize both the serum IgG clonal distribution and the anti-PF4 clonal distribution.

FIGS. 3A and 3B. The difference in resolution for the detection of M-proteins and micro M-proteins in a patient with an IgG kappa monoclonal gammopathy of undetermined significance (MGUS) and a patient without MGUS. FIG. 3A shows M-proteins detected using Mass-Fix from a patient with a clinically defined Ig kappa monoclonal (top) and a patient that is negative for an M-protein (bottom). FIG. 3B shows high-resolution mass spectra of the same patients—a patient with a clinically defined Ig kappa monoclonal (top) and a patient that is negative for an M-protein (bottom). Clones undetectable by the mass fix assay become apparent on higher resolution assays (“micro m-proteins”).

FIGS. 4A-4G. FIG. 4A) A 65-year-old male patient (Patient 1, PT1) with multiple thrombosis was found to have an IgG Kappa monoclonal antibody on Mass-Fix assay. The distribution of lambda, kappa containing immunoglobulins (Igs), and the light chain distribution of kappa and lambda light chains associated with an IgG heavy chain are shown. The box represents the expected m/z for doubly charged light chains. FIG. 4B) Comparison of the IgG kappa light chain m/z spectra, mass of the serum M-protein and anti-PF4 antibody profile for the anti-IgG enrichment and the anti-kappa enrichment shows that the mass of the kappa light chain from the samples were almost identical at 23,375.4 and 23,375.8 Daltons, respectively. FIG. 4C) The IgG heavy chain spectra show multiple peaks that represent multiply glycosylated forms of the monoclonal antibody, also showing almost identical masses from serum and eluate samples. FIG. 4D) Light chain fragmentation patterns of the serum M-protein vs the anti-PF4 antibody. Circles represent the common fragmentation ions between the two fragmentation studies. FIG. 4E) Immunofixation electrophoresis of patient serum, serum purified over PF4-treated Heparin Sepharose® beads, and over PBS-treated Heparin Sepharose® beads is shown. FIG. 4F) PEA results from PF4 vs PBS Heparin Sepharose® eluates. FIG. 4G) PF4-PVS ELISA results from PF4 vs PBS Heparin Sepharose® eluates. Means and +1 standard deviation are shown (n=3).

FIGS. 5A-5D. FIG. 5A) PT2 with spontaneous HIT does not have an M protein based on Mass-Fix testing. The distribution of lambda, kappa containing immunoglobulins (Igs), and the light chain distribution of kappa and lambda light chains associated with an IgG heavy chain are shown. The box represents the expected m/z for doubly charged light chains. FIG. 5B) Ig light chain+11 mass/charge distributions of PT2 demonstrating the mass similarities between the patient's overexpressed IgG kappa micro M-protein and the patient's anti-PF4 antibody (upper tables). Left: The IgG light chain distribution for the patient's serum obtained from nanobody enrichment with anti-lambda, anti-kappa and anti-IgG are shown. The serum demonstrated multiple IgG kappa and IgG lambda micro M-proteins expressed above the polyclonal background. The arrow points to an overexpressed IgG kappa micro M-protein with characteristics consistent with an anti-PF4 IgG kappa identified. Right—Ig light chain m/z distribution for the anti-PF4 antibodies from the nanobody enrichment with anti-kappa and anti-IgG; lambda spectra did not contain IgG. FIG. 5C) PEA results from PF4 vs PBS Heparin Sepharose® eluates and. FIG. 5D) PF4-PVS ELISA results from PF4 vs PBS Heparin Sepharose® eluates. Means and +1 standard deviation are shown (n=3).

FIGS. 6A-6D. FIG. 6A) PT3 with spontaneous HIT does not have an M protein based on Mass-Fix testing. The distribution of lambda, kappa containing immunoglobulins (Igs), and the light chain distribution of kappa and lambda light chains associated with an IgG heavy chain are shown. The box represents the expected m/z for doubly charged light chains. FIG. 6B) Ig light chain+11 mass/charge distributions of PT3 demonstrating the mass similarities between the patient's overexpressed IgG kappa micro M-protein and the patient's anti-PF4 antibody (upper tables). Left: The IgG light chain distribution for the patient's serum obtained from nanobody enrichment with anti-lambda, anti-kappa and anti-IgG are shown. The serum demonstrated multiple IgG kappa and IgG lambda micro M-proteins expressed above the polyclonal background. The arrow points to an overexpressed IgG kappa micro M-protein with characteristics consistent with an anti-PF4 IgG kappa identified. Right—Ig light chain m/z distribution for the anti-PF4 antibodies from the nanobody enrichment with anti-kappa and anti-IgG. FIG. 6C) PEA results from PF4 vs PBS Heparin Sepharose® eluates. FIG. 6D) PF4-PVS ELISA results from PF4 vs PBS Heparin Sepharose® eluates. Means and +1 standard deviation are shown (n=3).

FIGS. 7A-7D. FIG. 7A) PT4 does not have an M protein based on Mass-Fix testing. The distribution of lambda, kappa containing immunoglobulins (Igs), and the light chain distribution of kappa and lambda light chains associated with an IgG heavy chain are shown. The box represents the expected m/z for doubly charged light chains. FIG. 7B) Ig Light Chain+11 mass/charge distributions of a PT4 demonstrating the mass similarities between the patient's overexpressed IgG lambda micro M-protein and the patient's anti-PF4 antibody (upper tables). Left: The IgG light chain distribution for the patient's serum obtained from nanobody enrichment with anti-lambda, anti-kappa and anti-IgG are shown. The serum demonstrated multiple IgG kappa and IgG lambda micro M-proteins expressed above the polyclonal background. The arrows point to a couple of overexpressed IgG lambda micro M-protein with characteristics consistent with anti-PF4 IgG lambdas identified. Right—Ig light chain m/z distribution for the anti-PF4 antibodies from the nanobody enrichment with anti-IgG. FIG. 7C) PEA results from PF4 vs PBS Heparin Sepharose® eluates. FIG. 7D) PF4-PVS ELISA results from PF4 vs PBS Heparin Sepharose® eluates. Means and +1 standard deviation are shown (n=3).

FIGS. 8A-8D. FIG. 8A) PT5 has an M protein (IgG Lambda) based on Mass-Fix testing. The distribution of lambda, kappa containing immunoglobulins (Igs), and the light chain distribution of kappa and lambda light chains associated with an IgG heavy chain are shown. The box represents the expected m/z for doubly charged light chains. FIG. 8B) Ig Light Chain+11 mass/charge distributions of PT5 demonstrating the mass similarities between the patient's overexpressed IgG lambda M-protein and the patient's anti-PF4 antibody (upper tables). Left: The IgG light chain distribution for the patient's serum obtained from nanobody enrichment with anti-lambda, anti-kappa and anti-IgG are shown. The serum demonstrated multiple IgG kappa and IgG lambda micro M-proteins and the IgG lambda M-protein expressed above the polyclonal background. The arrow points to the IgG lambda M protein with characteristics consistent with an anti-PF4 IgG lambda identified. Right—Ig light chain m/z distribution for the anti-PF4 antibodies from the nanobody enrichment with anti-IgG. FIG. 8C) PEA results from PF4 vs PBS Heparin SepharoseR eluates. FIG. 8D) PF4-PVS ELISA results from PF4 vs PBS Heparin Sepharose® eluates. Means and +1 standard deviation are shown (n=3).

FIGS. 9A-9D. FIG. 9A) PT6 has a small M protein (IgG Lambda) based on Mass-Fix testing. The distribution of lambda, kappa containing immunoglobulins (Igs), and the light chain distribution of kappa and lambda light chains associated with an IgG heavy chain are shown. The box represents the expected m/z for doubly charged light chains. FIG. 9B) Ig light chain+11 mass/charge distributions of PT6 demonstrating the mass similarities between the patient's overexpressed IgG kappa micro M-proteins and the patient's anti-PF4 antibody (upper tables). Left: The IgG light chain distribution for the patient's serum obtained from nanobody enrichment with anti-lambda, anti-kappa and anti-IgG are shown. The serum demonstrated multiple IgG kappa and IgG lambda micro M-proteins and the IgG lambda M-protein expressed above the polyclonal background. The arrows point to the IgG kappa micro M proteins with characteristics consistent with anti-PF4 IgG kappa antibodies identified. Right—Ig light chain m/z distribution for the anti-PF4 antibodies from the nanobody enrichment with anti-IgG; lambda spectra did not contain IgG. FIG. 9C) PEA results from PF4 vs PBS Heparin Sepharose® eluates. FIG. 9D) PF4-PVS ELISA results from PF4 vs PBS Heparin Sepharose® eluates. Means and +1 standard deviation are shown (n=3).

FIGS. 10A-10C. FIG. 10A) Ig light chain+11 mass/charge distributions of PT7 demonstrating no mass similarities between the patient's few overexpressed IgG kappa and lambda micro M-proteins and the patients anti-PF4 antibody (no monoclonal/oligoclonal antibodies noted). Left: The IgG light chain distribution for the patient's serum and PF4 eluate obtained from nanobody enrichment with anti-lambda, anti-kappa, and anti-IgG are shown. No antibodies were found on PF4 immunoenrichment. FIG. 10B) PEA results from PF4 vs PBS Heparin Sepharose® eluates. FIG. 10C) PF4-PVS ELISA results from PF4 vs PBS Heparin Sepharose® eluates. Means and +1 standard deviation are shown (n=3).

FIG. 11. Serum from two monoclonal gammopathy of undetermined significance (MGUS) patient controls without suspected anti-PF4 antibodies was enriched with anti-IgG (C1-Gamma and C2-Gamma), anti-kappa (C1-Kappa), or anti-lambda (C2-Lambda) nanobodies. The two patients were also enriched for anti-PF4 antibodies followed by enrichment with anti-IgG (PF4-Gamma), anti-kappa (PF4-Kappa), and anti-lambda (PF4-Lambda). The results demonstrate no non-specific binding of the M-protein to the PF4 enrichment beads.

FIGS. 12A-12E. FIG. 12A) Ig light chain+11 mass/charge distributions of VITT/TTS Patient 1 (vaccine-induced immune thrombotic thrombocytopenia/Thrombosis & Thrombocytopenia Syndrome) demonstrating mass similarities between the patient's overexpressed IgG lambda micro M-protein and the patient's anti-PF4 antibody. Left: The IgG light chain distribution for the patient's serum obtained from nanobody enrichment with anti-lambda, anti-kappa and anti-IgG are shown. The serum demonstrated multiple IgG kappa and IgG lambda micro M-proteins expressed above the polyclonal background. The arrow points to an overexpressed IgG lambda micro M-protein with characteristics consistent with an anti-PF4 IgG lambda identified. Right—Ig light chain m/z distribution for the anti-PF4 antibodies from the nanobody enrichment with anti-lambda and anti-IgG; kappa spectra did not contain IgG. FIG. 12B) Ig light chain+11 mass/charge distributions of VITT/TTS Patient 2 demonstrating mass similarities between two (biclonal) of the patient's overexpressed IgG lambda micro M-protein and the patient's anti-PF4 antibody. Left: The IgG light chain distribution for the patient's serum obtained from nanobody enrichment with anti-lambda, anti-kappa and anti-IgG are shown. The serum demonstrated multiple IgG kappa and IgG lambda micro M-proteins expressed above the polyclonal background. The arrows point to two overexpressed IgG lambda micro M-proteins with characteristics consistent with the two anti-PF4 IgG lambdas identified. Right—Ig light chain m/z distribution for the anti-PF4 antibodies from the nanobody enrichment with anti-lambda and anti-IgG; kappa spectra did not contain IgG. FIG. 12C) Ig light chain+11 mass/charge distributions of VITT/TTS Patient 3 demonstrating mass similarities between three (triclonal) of the patient's overexpressed IgG lambda micro M-protein and the patient's anti-PF4 antibody. Left: The IgG light chain distribution for the patient's serum obtained from nanobody enrichment with anti-lambda, anti-kappa and anti-IgG are shown. The serum demonstrated multiple IgG kappa and IgG lambda micro M-proteins expressed above the polyclonal background. Overexpressed IgG lambda micro M-protein with characteristics consistent with three anti-PF4 IgG lambda were identified. Right—Ig light chain m/z distribution for the anti-PF4 antibodies from the nanobody enrichment with anti-lambda and anti-IgG; kappa spectra did not contain IgG. FIG. 12D) Evaluation of the eluate from heparin-sepharose beads (PBS treated instead of PF4) revealed no antibodies. FIG. 12E) PEA and PF4-PVS ELISA results from PF4 vs PBS Heparin Sepharose® eluates for VITT/TTS Patients 1 and 2 (inadequate sample for VITT/TTS Patient 3 to perform this study) are presented. Means and +1 standard deviation are shown (n=3).

### DETAILED DESCRIPTION

This document provides methods and materials for identifying and/or treating mammals (e.g., humans) having, or at risk of developing, MGTS. In some cases, this document provides methods and materials for assessing a sample obtained from a mammal (e.g., a human) for the presence or absence of a population of monoclonal antibodies specific for a PF4 polypeptide. For example, a sample obtained from a mammal (e.g., a human) having one or more thrombotic and/or thrombocytopenic conditions can be assessed for the presence or absence of a population of monoclonal antibodies specific for a PF4 polypeptide as provided herein to identify the mammal as having, or as being at risk of developing, MGTS. In some cases, this document provides methods and materials for assessing a sample obtained from a mammal (e.g., a human) for the presence or absence of a population of oligoclonal antibodies specific for a PF4 polypeptide. For example, a sample obtained from a mammal (e.g., a human) having one or more thrombotic and/or thrombocytopenic conditions can be assessed for the presence or absence of a population of oligoclonal antibodies specific for a PF4 polypeptide as provided herein to identify the mammal as having, or as being at risk of developing, MGTS. This document also provides methods and materials for detecting a population of monoclonal or oligoclonal antibodies having binding specificity for a PF4 polypeptide in a sample obtained from a mammal (e.g., a human). For example, a sample obtained from a mammal (e.g., a human) can be assessed for the presence or absence of a population of monoclonal or oligoclonal antibodies having binding specificity for a PF4 polypeptide using mass spectrometry techniques. In some cases, the presence or absence of a population of monoclonal or oligoclonal antibodies having binding specificity for a PF4 polypeptide in a sample obtained from a mammal (e.g., a human such as a human having one or more thrombotic and/or thrombocytopenia conditions) can be used to identify the mammal as having MGTS. This document also provides methods and materials for treating a mammal (e.g., a human) identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from the mammal).

In some cases, a mammal (e.g., human) having any thrombotic/thrombocytopenic condition can be assessed and/or treated as described herein. Examples of thrombotic and/or thrombocytopenic conditions include, without limitation, HIT, HIT with thrombosis, delayed-onset HIT, flush-related HIT, persistent HIT, HIT with disseminated intravascular coagulation, refractory HIT, and spontaneous HIT (e.g., spontaneous HIT with thrombosis and spontaneous HIT without thrombosis).

Any appropriate mammal (e.g., a mammal having one or more thrombotic and/or thrombocytopenia conditions) can be identified as having, or as being at risk of developing, MGTS. In some cases, a mammal having one or more thrombotic and/or thrombocytopenic conditions can be a mammal that was administered an anticoagulant (e.g., heparin). In some cases, a mammal having one or more thrombotic and/or thrombocytopenia conditions can be a mammal that was not administered an anticoagulant (e.g., heparin). In some cases, a mammal having one or more thrombotic and/or thrombocytopenia conditions can be a mammal having one or more viral and/or bacterial illness prodrome. In some cases, a mammal having one or more thrombotic and/or thrombocytopenia conditions can be a mammal that underwent an orthopedic surgery. In some cases, a mammal having one or more thrombotic and/or thrombocytopenia conditions can be a mammal that was administered one or more vaccinations (e.g., an anti-coronavirus vaccination). Examples of mammals that can have one or more thrombotic and/or thrombocytopenia conditions and can be identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from a mammal) include, without limitation, primates (e.g., humans and monkeys), dogs, cats, horses, cows, pigs, sheep, rabbits, mice, and rats. For example, humans having one or more thrombotic and/or thrombocytopenia conditions can be identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from a mammal). For example, humans that were administered an anti-coronavirus vaccination and having one or more thrombotic and/or thrombocytopenia conditions can be identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from a mammal).

This document also provides methods and materials for detecting a population of monoclonal or oligoclonal antibodies having binding specificity for a PF4 polypeptide in a sample obtained from a mammal (e.g., a mammal such as a human having one or more thrombotic and/or thrombocytopenic conditions). For example, a sample obtained from a mammal (e.g., a human) having one or more thrombotic and/or thrombocytopenia conditions can be assessed for the presence or absence of a population of monoclonal or oligoclonal antibodies specific for a PF4 polypeptide. In some cases, a population of monoclonal or oligoclonal antibodies having binding specificity for a PF4 polypeptide in a sample obtained from a mammal can be detected above a polyclonal background of antibodies in the sample using mass spectroscopy. For example, the presence of one or more peaks on a mass spectrum from a sample can be used to detect the presence of a population of monoclonal or oligoclonal antibodies having binding specificity for a PF4 polypeptide in the sample.

A population of monoclonal antibodies having binding specificity for a PF4 polypeptide or a population of oligoclonal antibodies having binding specificity for a PF4 polypeptide can bind any appropriate PF4 polypeptide. Examples of PF4 polypeptides that a population of monoclonal antibodies or a population of oligoclonal antibodies can have binding specificity for include, without limitation, those set forth in the National Center for Biotechnology Information (NCBI) databases at, for example, accession no. NP_002610 (version NP_002610.1), accession no. AAA60067 (version AAA60067.1), accession no. AAA60066 (version AAA60066.1).

Any appropriate sample can be used to determine if a mammal (e.g., a human) having one or more thrombotic and/or thrombocytopenia conditions has a population of monoclonal antibodies or a population of oligoclonal antibodies specific for a PF4 polypeptide. For example, blood samples (e.g., whole blood samples, serum samples, and plasma samples) or urine samples can be obtained from a mammal being tested and can be assessed for the presence or absence of a population of monoclonal antibodies or a population of oligoclonal antibodies specific for a PF4 polypeptide. In some cases, a sample can be processed (e.g., to purify and/or isolate immunoglobulins).

In some cases, a sample can be treated (e.g., to remove components that could interfere with the mass spectrometry technique). For example, a sample can be treated to remove components other than antibodies (e.g., albumin polypeptides, non-IgG immunoglobulins, PF4 polypeptides, RNA, DNA, transferrin, complement proteins, alpha-2 macroglobulin polypeptides, hemoglobin, and fibrinogen)

In some cases, antibodies can be isolated from the samples or enriched (i.e. concentrated) in a sample. For example, a sample can be enriched or purified using immunopurification, centrifugation, filtration, ultrafiltration, dialysis, ion exchange chromatography, size exclusion chromatography, protein A/G affinity chromatography, affinity purification, precipitation, gel electrophoresis, capillary electrophoresis, chemical fractionation (e.g., antibody purification kits, such as Melon Gel Purification), and/or aptamer techniques. Immunopurification can include contacting a sample with an affinity matrix including a capture moiety, such as an antibody (e.g. single domain antibody fragments, also referred to as nanobodies) or an antigen, that is covalently attached to a solid phase (e.g., beads such as agarose beads) and eluting the captured antibodies to obtain an immunopurified sample. For example, immunopurification can result in enrichment of one or more antibodies isotypes (e.g., IgG, IgM, IgA, IgE, and IgD) and/or antibodies having a particular light chain type (e.g., kappa light chains and lambda light chains). In some cases, immunopurification can separate or enrich IgG antibodies in a sample. In some cases, immunopurification can separate or enrich IgM antibodies in a sample. In some cases, immunopurification can separate or enrich antibodies having kappa light chains in a sample. In some cases, immunopurification can separate or enrich antibodies having lambda light chains in a sample. For example, a sample obtained from a mammal (e.g., a mammal such as a human having one or more thrombotic and/or thrombocytopenia conditions) can be contacted with beads (e.g., agarose beads) containing nanobodies (e.g., camelid-derived nanobodies) directed against the constant domains of IgG antibodies, kappa light chains, or lambda light chains, and the captured antibodies can be eluted from the beads to obtain an immunopurified sample.

In some cases, immunopurification can separate or enrich antibodies having specificity for a PF4 polypeptide. For example, a sample obtained from a mammal (e.g., a mammal such as a human having one or more thrombotic and/or thrombocytopenia conditions) can be contacted with a solid substrate (e.g., beads such as agarose beads, platelets, and white blood cells) containing PF4 polypeptides (e.g., treated with PF4 polypeptides or containing immobilized PF4 polypeptides), and the captured antibodies having specificity for a PF4 polypeptide can be eluted from the solid substrate to obtain an immunopurified sample. In some cases, a sample obtained from a mammal (e.g., a mammal such as a human having one or more thrombotic and/or thrombocytopenia conditions) can be contacted with agarose beads containing PF4 polypeptides (e.g., Heparin Sepharose® beads treated with PF4 polypeptides), and the captured antibodies having specificity for a PF4 polypeptide can be eluted from the beads to obtain an immunopurified sample. In some cases, a solid substrate can contain PF4 polypeptides complexed a polyanion (e.g., heparin). Any solution can be used to elute captured antibodies having specificity for a PF4 polypeptide from a solid substrate. In some cases, a salt solution (e.g., a high salt solution) can be used to elute captured antibodies having specificity for a PF4 polypeptide from a solid substrate. In some cases, an acid can be used to elute captured antibodies having specificity for a PF4 polypeptide from a solid substrate.

In some cases, an immunopurified sample can be substantially pure. The term “substantially pure” as used herein with reference to an immunopurified sample means the sample is substantially free of other polypeptides, lipids, carbohydrates, and nucleic acid that are present in the sample prior to immunopurification. Thus, an immunopurified sample that is substantially pure can be an immunopurified sample that is at least 60 percent antibodies. In some cases, an immunopurified sample that is substantially pure can be at least about 65, 70, 75, 80, 85, 90, 95, or 99 percent antibodies.

In some cases, a sample can be processed to disassociate the light chains from heavy chains of antibodies present within the sample. Any appropriate method can be used to disassociate a light chain from a heavy chain of an antibody. For example, an antibody (e.g., a sample containing an antibody) can be contacted with a reducing agent. Examples of reducing agents that can be used to disassociate a light chain from a heavy chain of an antibody include, without limitation, dithiothreitol (DTT), tris(2-carboxyethyl)phosphine (TCEP), and beta-mercaptoethanol.

Any appropriate technique can be used to assess a sample obtained from a mammal (e.g., a mammal such as a human having one or more thrombotic and/or thrombocytopenic conditions) for the presence or absence of a population of monoclonal antibodies (e.g., autoantibodies) or a population of oligoclonal antibodies (e.g., autoantibodies) specific for a PF4 polypeptide. For example, a mass spectrometry technique, immunofixation electrophoresis (IFE), and/or serum protein electrophoresis (SPEP) can be used to detect the presence or absence of a population of monoclonal antibodies or a population of oligoclonal antibodies specific for a PF4 polypeptide. In some cases, when a mass spectrometry technique is used to detect the presence or absence of a population of monoclonal antibodies or a population of oligoclonal antibodies specific for a PF4 polypeptide, the mass spectrometry technique can be a tandem mass spectrometry technique. Examples of mass spectrometry techniques that can be used as described herein (e.g., to detect the presence or absence of a population of monoclonal antibodies or a population of oligoclonal antibodies specific for a PF4 polypeptide) include, without limitation, quadrupole time-of-flight (Q-TOF) mass spectrometry (e.g., Q-TOF mass spectrometer using electrospray ionization (ESI-Q-TOF)), matrix-assisted laser desorption/ionization (MALDI)-TOF mass spectrometry, orbitrap mass spectrometry, and Mass-Fix. In some cases, when a mass spectrometry technique is used to assess a sample obtained from a mammal for the presence or absence of a population of monoclonal antibodies specific for a PF4 polypeptide or the presence or absence of a population of oligoclonal antibodies specific for a PF4 polypeptide is Mass-Fix, a sample can be processed to remove PF4 polypeptides (e.g., using immunodepletion, size exclusion chromatography, and/or dialysis) and the processed sample can be subjected to Mass-Fix.

In some cases, a mass spectrometry technique can be coupled with one or more physical separation techniques. For example, a sample can be subjected to one or more physical separation techniques prior to being subjected to one or more mass spectrometry techniques. Examples of physical separation techniques that can be coupled with a mass spectrometry techniques that can be used as described herein (e.g., to detect the presence or absence of a population of monoclonal antibodies specific for a PF4 polypeptide or the presence or absence of a population of oligoclonal antibodies specific for a PF4 polypeptide) include, without limitation, liquid chromatography (e.g., high-performance liquid chromatography, microflow chromatography, and nanoflow chromatography), gas chromatography, capillary electrophoresis, immunopurification, and ion exchange chromatography.

In some cases, a mass spectrometry technique (e.g., ESI-Q-TOF mass spectrometry) can be used to analyze the mass spectrum of a sample, e.g., the mass spectrum of the +11 charge state of the antibodies in the sample. For example, for a population of monoclonal antibodies or a population of oligoclonal antibodies specific for a PF4 polypeptide, the peaks (e.g., the charged ion peaks) can occur at from about 2020 m/z to about 2200 m/z. In some cases, a mass spectrum can include multiple peaks. For example, when a population of monoclonal antibodies specific for a PF4 polypeptide includes multiple (e.g., two, three, four, or more) different glycosylated forms of the monoclonal antibody, a mass spectrum can include multiple peaks where each peak represents a single glycosylated form of the monoclonal antibody.

In some cases, a mass spectrometry technique can include converting the peaks (e.g., the charged ion peaks) on a mass spectrum to a molecular mass. For example, charged ion peaks can be deconvoluted to accurate molecular mass using a software program (e.g., a Bio Tool Kit ver. 2.2 plug-in software).

In some cases, the presence or absence of a population of monoclonal antibodies specific for a PF4 polypeptide or the presence or absence of a population of oligoclonal antibodies specific for a PF4 polypeptide in a sample obtained from a mammal (e.g., a human) can be detected as described in Example 1.

In some cases, methods for assessing a sample obtained from a mammal (e.g., a human) for the presence or absence of a population of monoclonal antibodies specific for a PF4 polypeptide or for the presence or absence of a population of oligoclonal antibodies specific for a PF4 polypeptide as provided herein can be used to identify a mammal having one or more thrombotic and/or thrombocytopenia conditions as having, or as being at risk of developing, MGTS. For example, when a mammal (e.g., a human) having one or more thrombotic and/or thrombocytopenia conditions is identified as having a population of monoclonal antibodies specific for a PF4 polypeptide or a population of oligoclonal antibodies specific for a PF4 polypeptide, the mammal can be classified as having, or as being at risk of developing, MGTS. For example, when a mammal (e.g., a human) having one or more thrombotic and/or thrombocytopenia conditions is identified as lacking a population of monoclonal antibodies specific for a PF4 polypeptide and lacking a population of oligoclonal antibodies specific for a PF4 polypeptide, the mammal can be classified as not having, or as not being at risk of developing, MGTS.

In some cases, methods for assessing a sample obtained from a mammal (e.g., a human) for the presence or absence of a population of monoclonal antibodies specific for a PF4 polypeptide or the presence or absence of a population of oligoclonal antibodies specific for a PF4 polypeptide as provided herein can be used to monitor a mammal having one or more thrombotic and/or thrombocytopenia conditions for the development of MGTS.

In some cases, methods for assessing a sample obtained from a mammal (e.g., a human) for the presence or absence of a population of monoclonal antibodies specific for a PF4 polypeptide or the presence or absence of a population of oligoclonal antibodies specific for a PF4 polypeptide as provided herein can be used to monitor a mammal having one or more thrombotic and/or thrombocytopenia conditions and having, or at risk of developing, MGTS for the severity and/or progression of the MGTS.

In some cases, methods for assessing a sample obtained from a mammal (e.g., a human) for the presence or absence of a population of monoclonal antibodies specific for a PF4 polypeptide or the presence or absence of a population of oligoclonal antibodies specific for a PF4 polypeptide as provided herein can include determining whether the population of monoclonal antibodies specific for a PF4 polypeptide, when present, have platelet activating activity. For example, when a population of monoclonal antibodies specific for a PF4 polypeptide is detected in a sample obtained from a mammal (e.g., a human) having one or more thrombotic and/or thrombocytopenia conditions, the population of monoclonal antibodies specific for a PF4 polypeptide can be immunopurified and can be assessed for platelet activating activity. Any appropriate method can be used to assess a population of monoclonal antibodies or a population of oligoclonal antibodies specific for a PF4 polypeptide for platelet activating activity. For example, platelet activation assays using platelets treated with heparin can be used to assess a population of monoclonal antibodies specific for a PF4 polypeptide for platelet activating activity. For example, platelet activation assays using platelets treated with PF4 polypeptides can be used to assess a population of monoclonal antibodies specific for a PF4 polypeptide for platelet activating activity. In some cases, a platelet activation assay can be performed as described in Example 1.

In some cases, methods for assessing a sample obtained from a mammal (e.g., a human) for the presence or absence of a population of monoclonal antibodies specific for a PF4 polypeptide or the presence or absence of a population of oligoclonal antibodies specific for a PF4 polypeptide as provided herein can include determining whether the population of monoclonal antibodies or oligoclonal antibodies specific for a PF4 polypeptide, when present, can target (e.g., target and bind) a PF4 polypeptide and/or a PF4-polyanion complex. For example, when a population of monoclonal antibodies specific for a PF4 polypeptide is detected in a sample obtained from a mammal (e.g., a human) having one or more thrombotic and/or thrombocytopenia conditions, the population of monoclonal antibodies specific for a PF4 polypeptide can be immunopurified and can be assessed for binding activity with a PF4 polypeptide and/or a PF4-polyanion complex. Any appropriate method can be used to assess a population of monoclonal antibodies specific for a PF4 polypeptide for binding activity with a PF4 polypeptide and/or a PF4-polyanion complex. For example, binding assays can be used to assess a population of monoclonal antibodies specific for a PF4 polypeptide for binding activity with a PF4 polypeptide. For example, binding assays can be used to assess a population of monoclonal antibodies specific for a PF4 polypeptide for binding activity with a PF4-polyanion complex. In some cases, a binding assay can be performed as described in Example 1.

This document also provides methods and materials for treating a mammal (e.g., a human) having one or more thrombotic and/or thrombocytopenic conditions, where one or more treatments are selected based on whether the mammal is identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from a mammal). For example, a sample obtained from a mammal (e.g., a mammal such as a human having one or more thrombotic and/or thrombocytopenia conditions) can be assessed for the presence or absence of a population of monoclonal antibodies having binding specificity for a PF4 polypeptide, and one or more treatments can be selected and, optionally, administered, to the mammal based, at least in part, on whether or not the population of monoclonal antibodies having binding specificity for a PF4 polypeptide is detected. For example, a sample obtained from a mammal (e.g., a mammal such as a human having one or more thrombotic and/or thrombocytopenia conditions) can be assessed for the presence or absence of a population of oligoclonal antibodies having binding specificity for a PF4 polypeptide, and one or more treatments can be selected and, optionally, administered, to the mammal based, at least in part, on whether or not the population of oligoclonal antibodies having binding specificity for a PF4 polypeptide is detected.

When a mammal (e.g., a human) having one or more thrombotic and/or thrombocytopenia conditions is identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from a mammal), the mammal can be administered, or instructed to self-administer, one or more therapies effective to treat a monoclonal gammopathy. Examples of therapies that can be used to treat a mammal having one or more thrombotic and/or thrombocytopenia conditions and identified as having, or as being at risk of developing, MGTS as described herein (e.g., based, at least in part, on the presence of a population of monoclonal antibodies having binding specificity for a PF4 polypeptide or the presence of a population of oligoclonal antibodies having binding specificity for a PF4 polypeptide in a sample obtained from a mammal) include, without limitation, B-cell targeted therapies, plasma cell targeted therapies, plasmapheresis, therapeutic plasma exchange, and intravenous immunoglobulin G.

When a mammal (e.g., a human) having one or more thrombotic and/or thrombocytopenia conditions is identified as not having, or as not being at risk of developing, MGTS as described herein (e.g., based, at least in part, on the absence of a population of monoclonal antibodies having binding specificity for a PF4 polypeptide and the absence of a population of oligoclonal antibodies having binding specificity for a PF4 polypeptide in a sample obtained from a mammal), the mammal can be administered, or instructed to self-administer, one or more therapies effective to prevent or treat thrombosis. Examples of therapies effective to treat a mammal having one or more thrombotic and/or thrombocytopenia conditions and identified as not having, or as not being at risk of developing, MGTS as described herein (e.g., based, at least in part, on the absence of a population of monoclonal antibodies having binding specificity for a PF4 polypeptide and the absence of a population of oligoclonal antibodies having binding specificity for a PF4 polypeptide in a sample obtained from a mammal) include, without limitation, thrombolytics, anticoagulants (e.g., direct oral anticoagulants (DOACs), direct thrombin inhibitors (DTIs), danaparoid, and fondaparinux), intravenous immunoglobulins, and therapeutic plasma exchange.

The invention will be further described in the following examples, which do not limit the scope of the invention described in the claims.

### EXAMPLES

### Example 1: Monoclonal Gammopathy of Thrombotic Thrombocytopenic Significance (MGTS) Due to Anti-PF4 Antibodies

This Example describes the discovery that some patients (e.g., HIT patients and spontaneous HIT patients) develop monoclonal or oligoclonal anti-PF4 antibodies that can be used to identify those patients have having a previously unidentified clinical entity referred to herein as Monoclonal Gammopathy of Thrombotic/Thrombocytopenia Significance (MGTS).

### Methods

### Patient Samples

Blood samples were from patients with HIT or spontaneous HIT.

### Anti-PF4 Antibody Enrichment

Heparin Sepharose® beads (0.3 mL, Cytiva Lifesciences) were incubated with 0.2 mL (0.2 mg) recombinant PF4 (Protein Foundry) at room temperature for 1 hour, washed with phosphate buffered saline, pH 7.4, and resuspended in a 0.5 mL volume. Anti-PF4 antibodies were enriched by incubating patient sera/plasma (0.5 mL) with 0.5 mL of the prepared PF4-heparin beads or control beads (PBS-treated Heparin Sepharose® beads) for 1 hour at room temperature. After washing, elution from the beads was performed with 0.5 mL 2M NaCl for 1 hour at room temperature. The eluate was then dialyzed against 1xPBS (pH 7.4). The eluate and native plasma/sera were evaluated in the PF4-PVS ELISA, SRA (serotonin release assay), and PEA (PF4-dependent P-selectin expression assay) and further characterized by mass spectrometry.

### PF4 Polyvinyl Sulfonate ELISA

ELISA plates (Thermo Scientific) were incubated with recombinant PF4 (Protein Foundry; 10 μg/mL) and Polyvinylsulfonate (PVS, Polysciences; 9 μg/mL). Plates were washed with phosphate buffered saline, pH 7.4 (PBS) 0.1% Tween and blocked with Superblock T20 (Thermo Scientific). Eluate samples were tested at a 1:10 dilution. Goat anti human IgG fc antibody (Jackson Immunoresearch) and pNPP (Sigma Aldrich) were used for colorimetric detection. Optical density was recorded at 405 nm.

### Functional Platelet Activation Assays

The PF4-dependent p-selectin expression assay (PEA) was performed as previously described (Samuelson-Bannow et al., Blood, 137(8): 1082-1089 (2021)). The serotonin release assay was performed as previously described (Sheridan et al., Blood, 67(1):27-30 (1986)) with minor modifications.

### M-Protein Detection MALDI-TOF MS (Mass-Fix) Assay

The method was performed as described elsewhere (Kohlhagen et al., Clin. Chem. Lab. Med., 59(1):155-163 (2020)). Briefly, a total of 50 μL of serum/plasma from each patient was immunoenriched using five separate (IgA, IgG, IgM, Kappa and Lambda) CaptureSelect™ nanobody resins (Thermo-Fisher Scientific, Waltham, MA). The pre-analytical processing of immunoenrichment, washing, reduction and elution were performed on a Hamilton Starlet liquid handler (Hamilton, Reno, NV). The resulting purified eluates were mixed with α-cyano-4-hydroxycinnamic acid (ACHA) matrix and spotted onto a Bruker MALDI-TOF plate using a ttpLabtech Mosquito nanoliter pipettor (sptlabtech, Hertfordshire, UK). Results were interpreted by both the laboratory technician and a laboratory director.

Detection of M-Proteins and Micro M-Proteins by LC-ESI-QTOF MS (miRAAM) in Serum and PF4 Enriched Eluates

The basic method was as described elsewhere (Barnidge et al., J. Proteome Res., 13(11):5198-5205 (2014); and Barnidge et al., J. Proteome Res., 13(3):1419-1427 (2014)). Briefly, serum/plasma or serum/plasma enriched for anti-PF4 antibodies were enriched using a 50:50 mix of camelid-derived nanobodies directed against the constant domains of IgG, kappa and lambda (Thermo Fisher Scientific). 10 μL of beads were incubated with 20 μL of serum or 50 μL of anti-PF4 antibody enriched eluate diluted into 180 μL of phosphate or 200 μL of buffered saline (PBS) for 45 minutes at ambient temperature. Subsequently, the supernatant was removed, and the beads were washed three times with 500 μL of water. Samples were eluted with 100 μL of 5% acetic acid and combined with 50 μL of 100 mM dithiothreitol (DTT) in 1M ammonium bicarbonate to disassociate Immunoglobulins into separated light chain (LC) and heavy chain (HC) components.

An Eksigent Ekspert 200 microLC (Framingham, MA) was used to separate immunoglobulin LCs prior to ionization and detection. The mobile phases included an aqueous phase A (100% water+1% formic acid) and an organic phase B (90% acetonitrile+10% isopropanol+0.1% formic acid). 5 μL of each bead elution was injected per analysis onto a Poroshell 300SB-C3 column (1.0 mm×75 mm) with a 5 μm particle size placed in a 60° C. column heater. The gradient used has been described previously and the flow rate was 25 μL/minute.

A SCIEX TripleTOF 5600 quadrupole time-of-flight (Q-TOF) mass spectrometer using electrospray ionization in positive ion mode was used for miRAMM analysis. Data analysis was performed using Analyst TF v1.8.1 and Peak View ver. 2.2. Over expressed Ig were inferred from the light chain+11 (m/z 2020 to m/z 2200) as described elsewhere (Barnidge et al., J. Proteome Res., 13(11):5198-5205 (2014); and Barnidge et al., J. Proteome Res., 13(3): 1419-1427 (2014)). The mass spectra of the multiply charged LC ions were deconvoluted to accurate molecular mass using the Bio Tool Kit ver. 2.2 plug-in software. The retention time of the monoclonal LC in each pre-treatment patient sample was tracked using Peak View. The instrument was calibrated every 5 samples using the automated calibrant delivery system (CDS). Mass measurement accuracy was estimated to be 15 ppm over the course of the analysis.

### Immunofixation Electrophoresis

Serum IFE was performed using Hydrasys 9IF gels (Sebia, Paris, France) following manufacturer's recommendations.

### Results

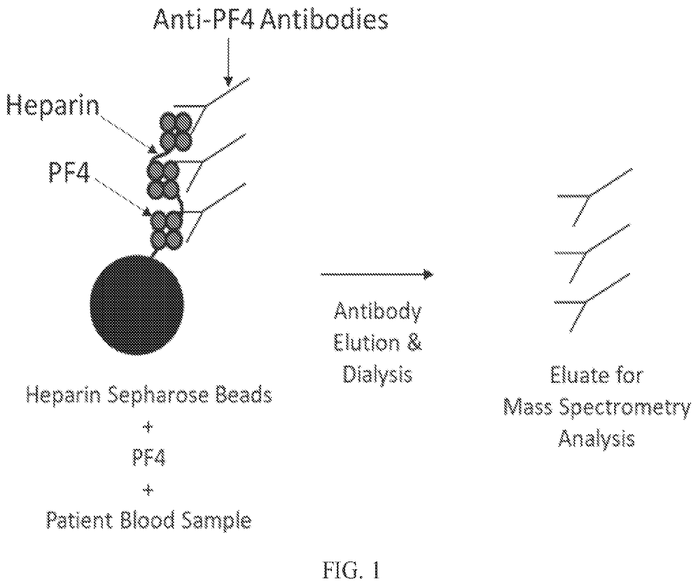

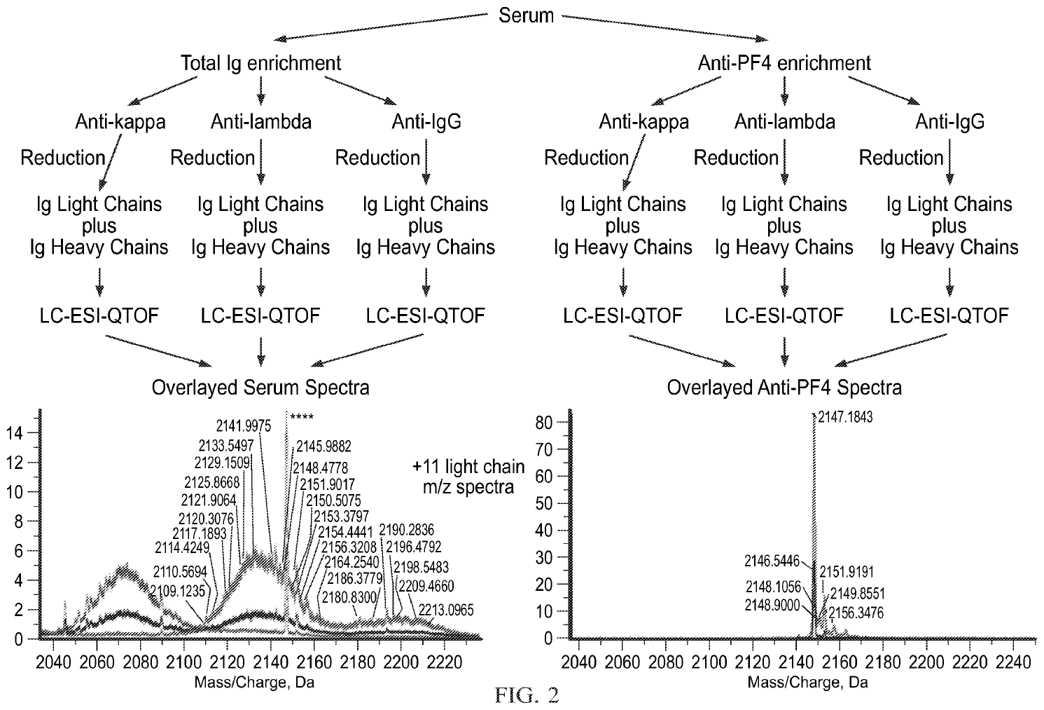

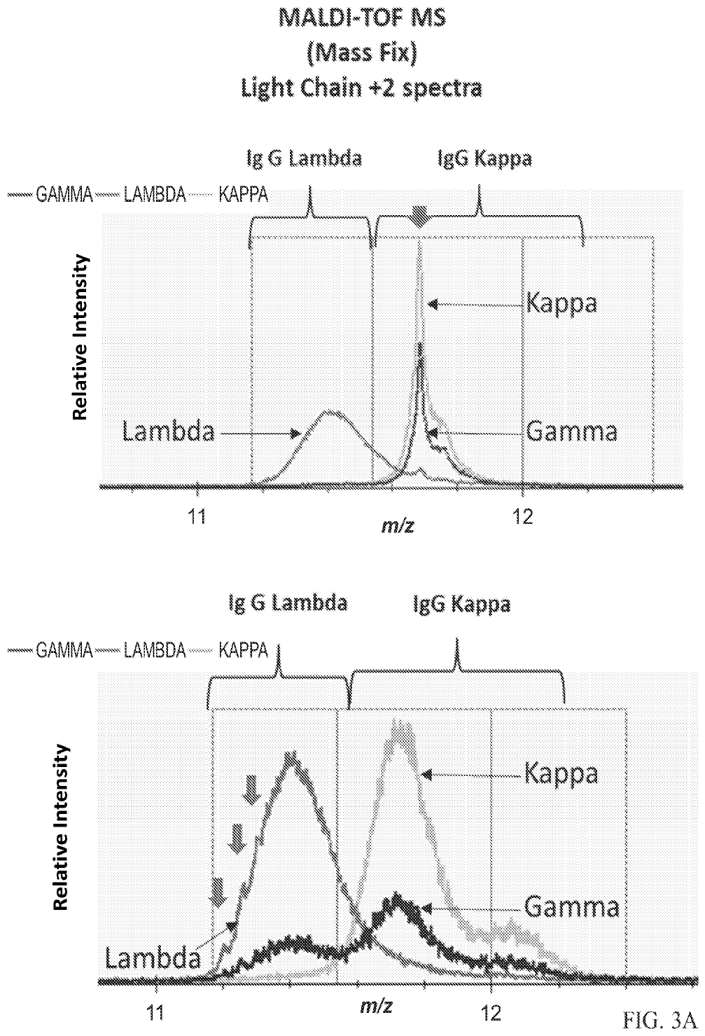

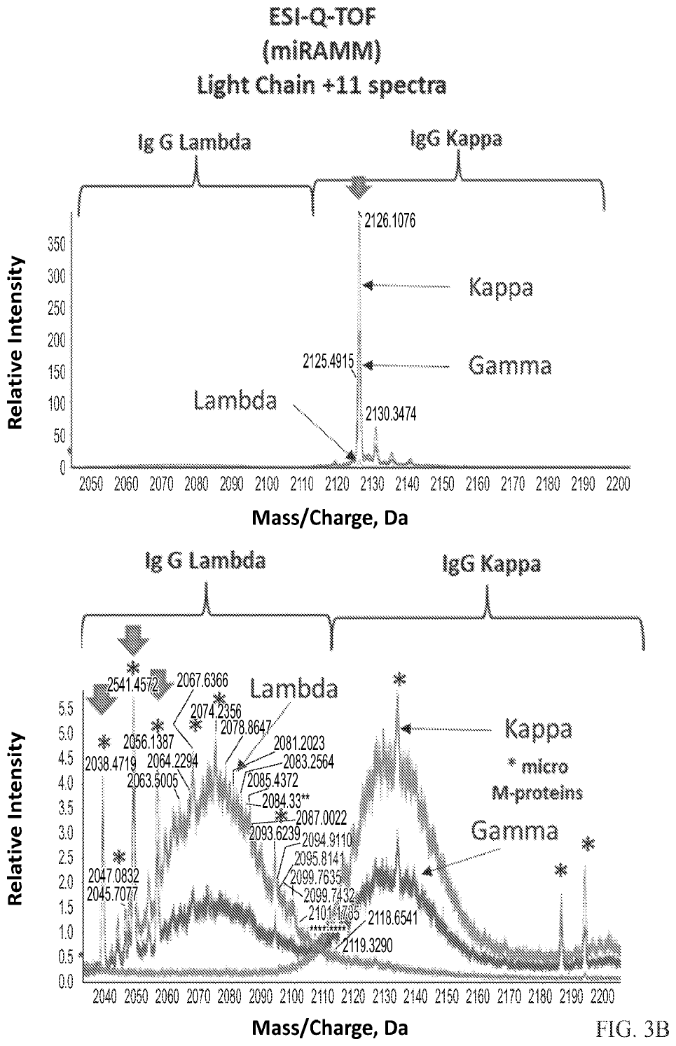

Anti-PF4 antibodies were enriched using Heparin Sepharose® beads treated with PF4 as shown in FIG. 1. The bound antibodies were eluted under high salt conditions and then dialyzed into PBS prior to downstream analysis by mass spectrometry (FIG. 1). The mass spectrometry studies performed are summarized schematically in FIG. 2. These studies sought to characterize the serum IgG and anti-PF4 eluate IgG clonal distribution. The multiple enrichment scheme allows for isotyping the anti-PF4 antibodies and to assess the level of anti-PF4 antibody production relative to the patient's polyclonal IgG background. FIG. 3 highlights the ability of the “mass-fix” mass spectrometry technique to document the presence of monoclonal antibodies (“M-proteins”; FIG. 3A, top). It does not however, detect “micro m-proteins” (FIG. 3A, bottom) that are detected by high-resolution mass spectrometry techniques (FIG. 3B).

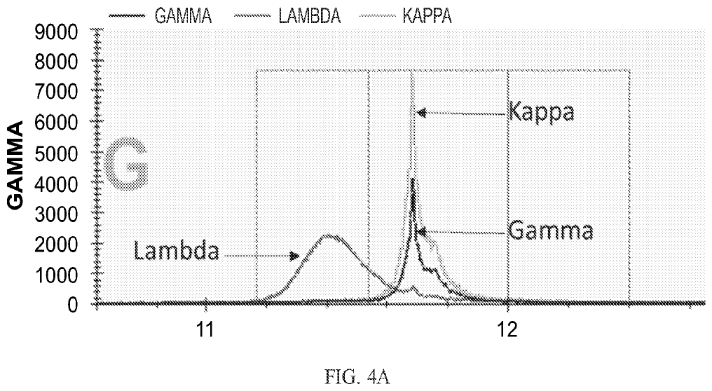

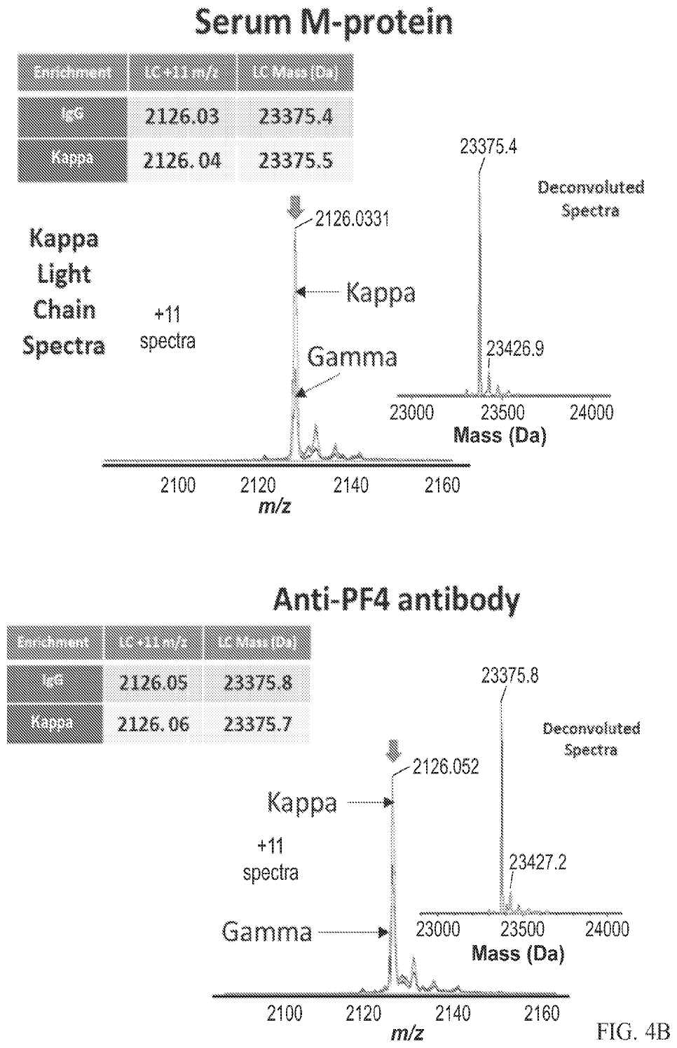

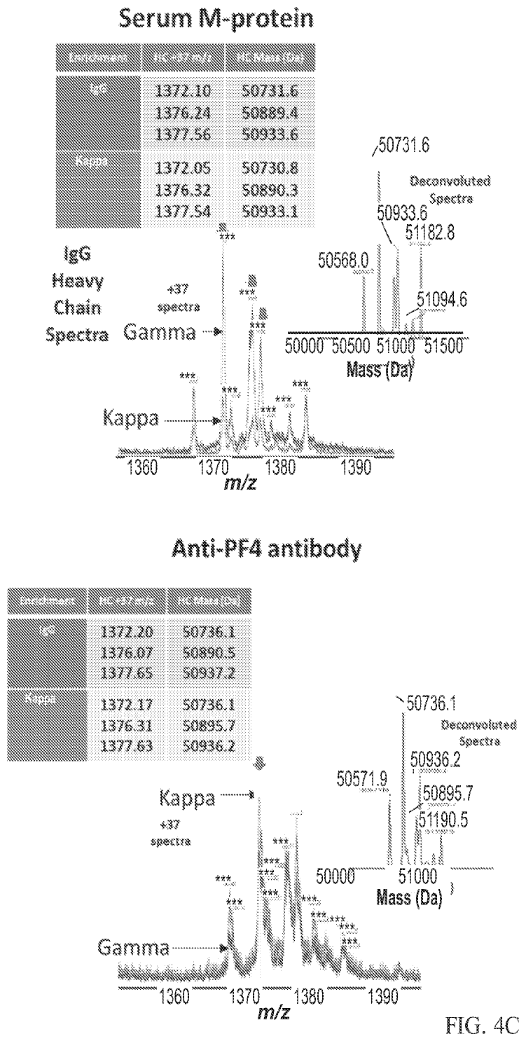

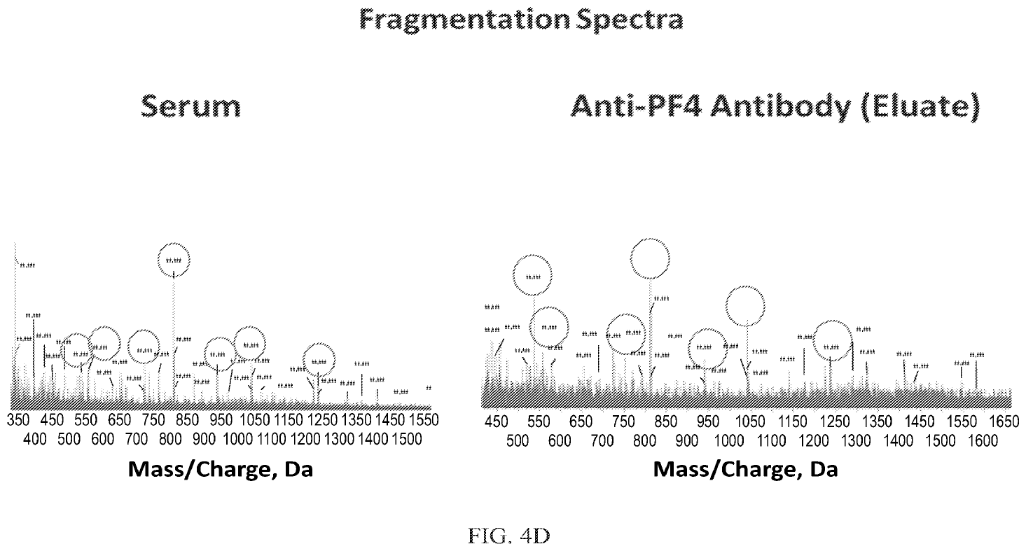

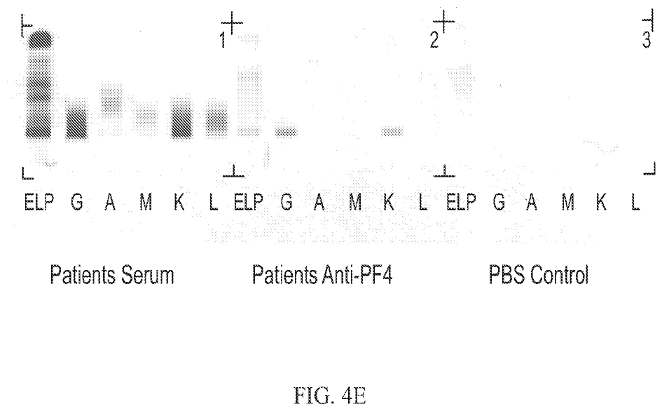

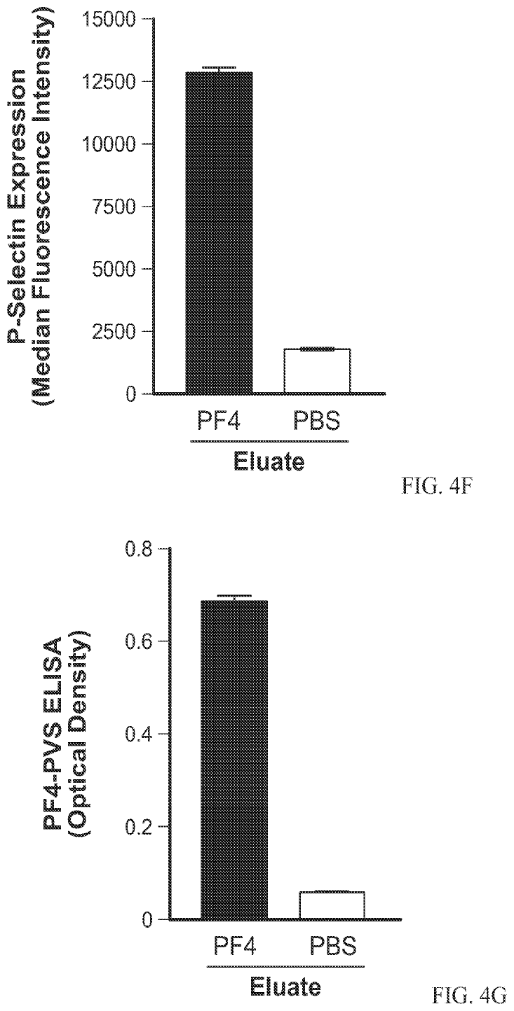

The index case (Patient 1, PT1) was a 65-year-old male with recurrent multiple thrombosis (deep venous thrombosis [DVT], pulmonary embolism, Liver and splenic thrombosis, coronary thrombosis) and thrombocytopenia and a history of positivity in HIT ELISAs and SRAs over a period of years. He was found to have an IgG Kappa monoclonal antibody on Mass-Fix assay (FIG. 4A). Comparison of the IgG kappa light chain m/z spectra and mass of the serum M-protein and anti-PF4 antibody profile from anti-IgG and anti-kappa enrichment showed that the mass of the kappa light chain from the samples were almost identical (FIG. 4B). The IgG heavy chain spectra showed multiple peaks that represent multiply glycosylated forms of the monoclonal antibody, also demonstrating almost identical masses from serum and PF4 Heparin Sepharose® Bead eluate samples (FIG. 4C). Light chain fragmentation patterns of the serum M-protein vs the anti-PF4 antibody demonstrates very similar fragmentation suggesting that the two antibodies are the same (FIG. 4D). Circles represent the common fragmentation ions between the two fragmentation studies. Standard Immunofixation electrophoresis of patient serum, serum purified over PF4-treated Heparin Sepharose® beads, and over PBS-treated Heparin Sepharose® beads is shown and demonstrates that the IgG kappa monoclonal is noted only in serum and eluate purified over PF4-heparin beads (FIG. 4E). Only PF4-heparin purified eluates demonstrate platelet-activation (FIG. 4F) and binding to PF4-PVS complexes in ELISA (FIG. 4G) as opposed to control Heparin Sepharose® beads demonstrating that the PF4 heparin bead eluate (which has the M-protein) has anti-PF4 antibody activity. SRA activity in the PF4 and control Heparin Sepharose® bead eluates, similarly, were at 57% vs 1%, respectively.

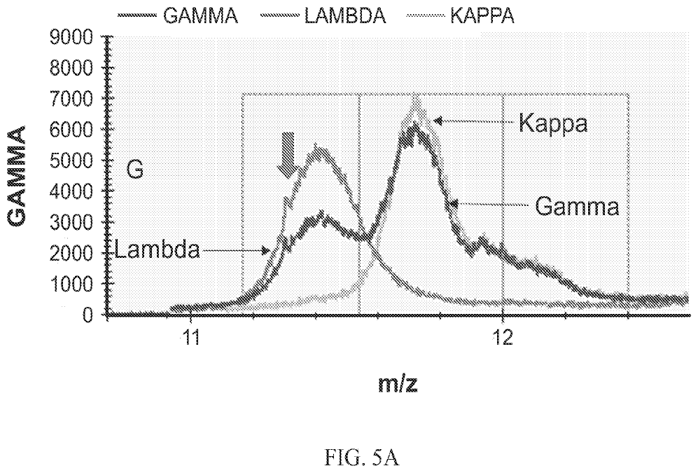

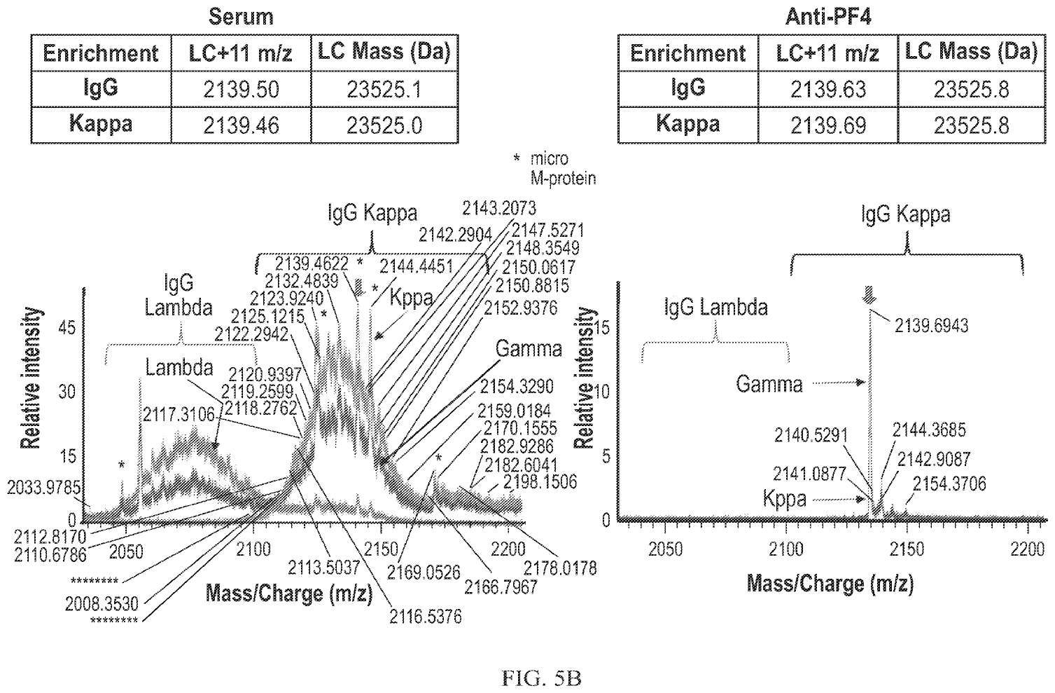

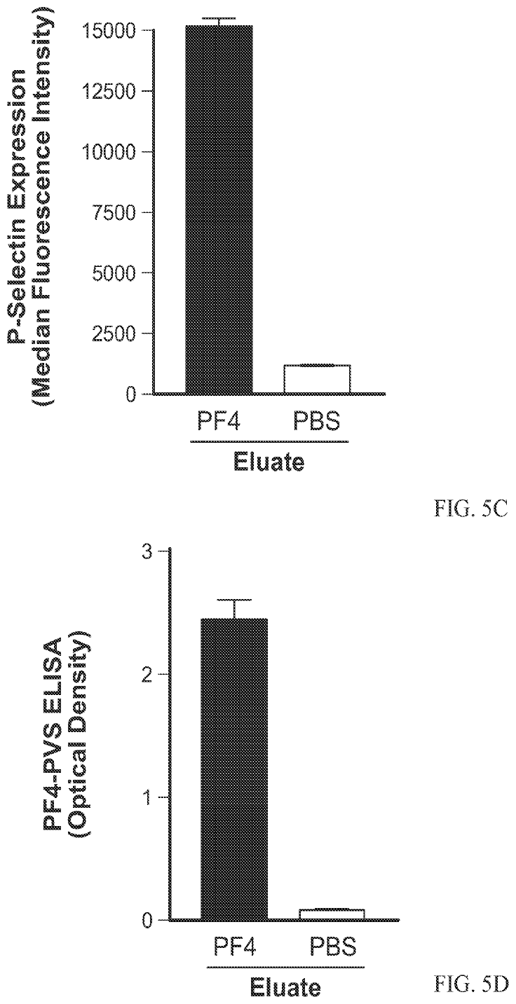

Patient 2 (PT2) was a 71-year-old female who developed multiple thrombosis and thrombocytopenia after knee arthroplasty and was diagnosed with spontaneous HIT. In contrast to PT1, Mass-Fix was negative for M-proteins (FIG. 5A). High-resolution mass spectrometry analysis of the serum demonstrated multiple micro M-protein with one IgG kappa clone demonstrated striking mass similarities with the patient's anti-PF4 antibody (upper tables) suggesting that the anti-PF4 antibody is a monoclonal antibody (FIG. 5B). FIG. 5C and FIG. 5D demonstrate that the PF4-heparin bead eluate, as opposed to control beads (PBS-treated heparin beads) has platelet activating activity and binding to PF4-PVS, respectively demonstrating that the PF4 heparin bead eluate (which has the monoclonal antibody) has anti-PF4 antibody activity.

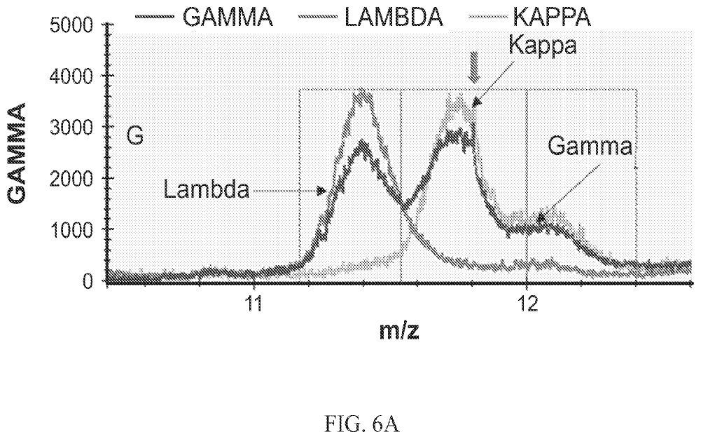

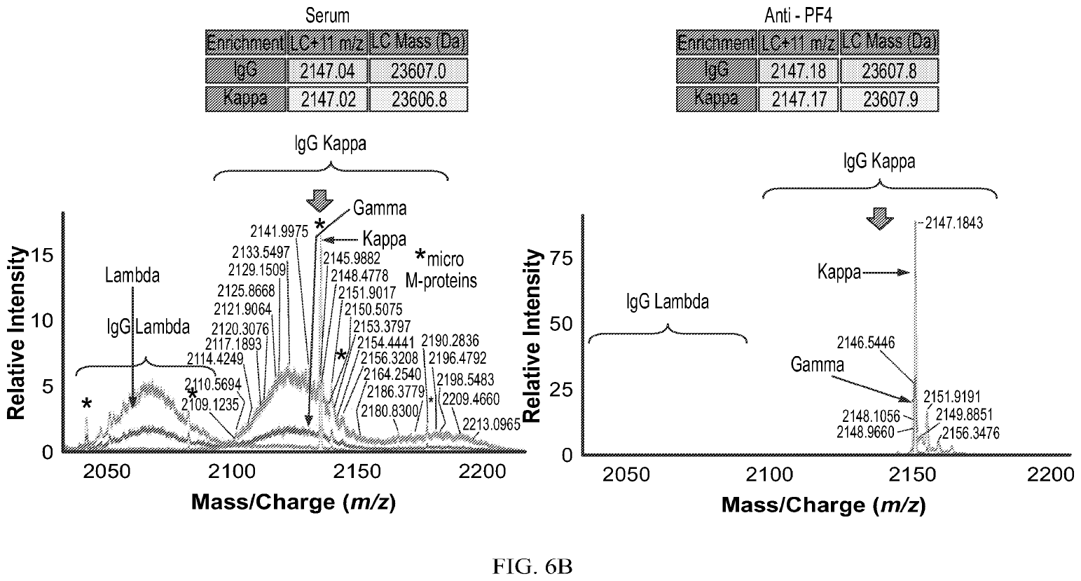

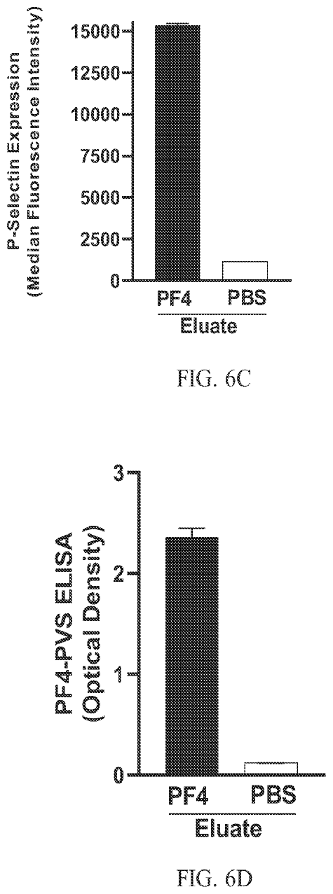

Patient 3 (PT3) was a 30-year-old male who presented with DVTs and cerebrovascular accident (thrombotic stroke) several days after a viral/bacterial prodrome-like illness characterized by nausea and fatigue and was diagnosed with spontaneous HIT several days later. He was strongly positive in HIT ELISA and SRA. Similar to PT2, Mass-Fix was negative for M-proteins (FIG. 6A). High-resolution mass spectrometry analysis demonstrated striking mass similarities between the patient's overexpressed IgG kappa micro M-protein and the patient's anti-PF4 antibody (upper tables) suggesting that the anti-PF4 antibody was a monoclonal antibody (FIG. 6B). FIG. 6C and FIG. 6D demonstrate that the PF4-heparin bead eluate, as opposed to control beads (PBS-treated heparin beads) has platelet activating activity and binding to PF4-PVS, respectively demonstrating that the PF4 heparin bead eluate (which has the monoclonal antibody) has anti-PF4 antibody activity.

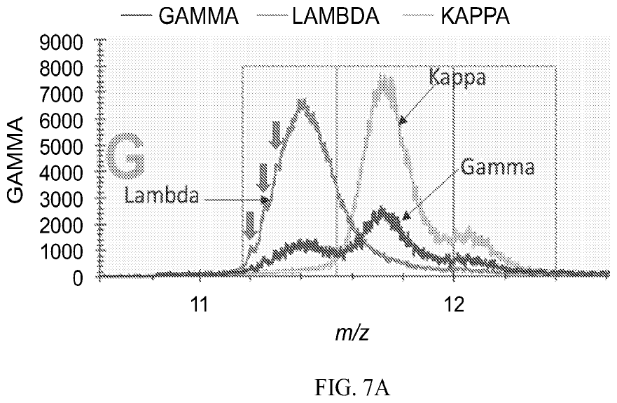

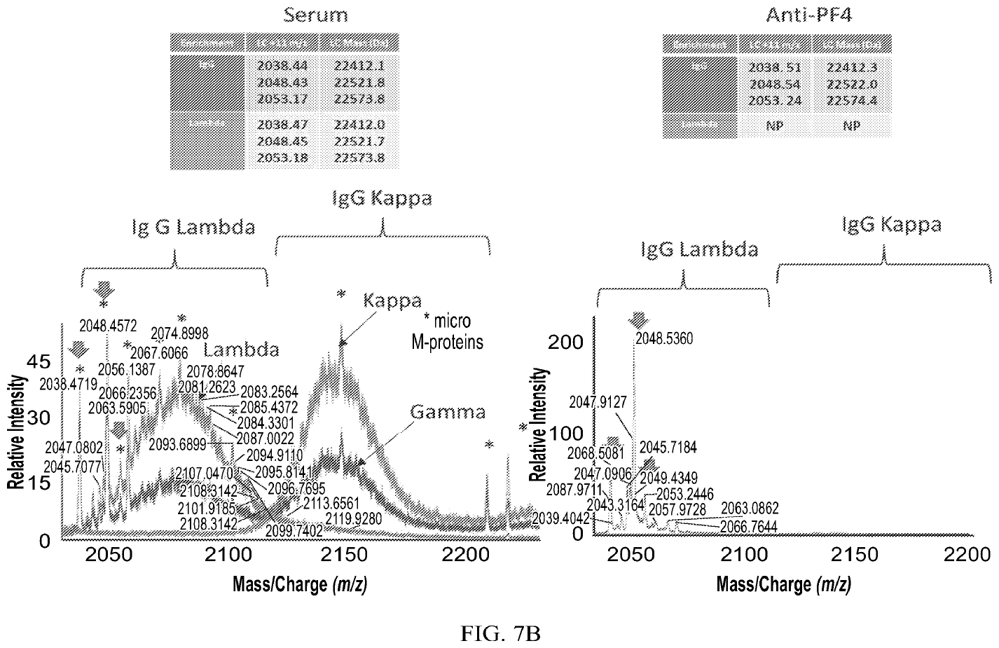

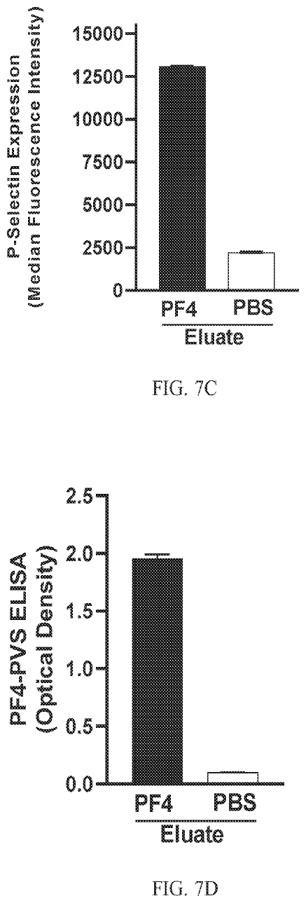

Patient 4 (PT4) was a 70-year-old male who underwent coronary artery bypass surgery and had intra- and post-operative heparin exposure. Four days after heparin treatment was ceased, he was noted to have bleeding from venipuncture sites, a platelet count of 10,000/μL and a DIC-like picture with elevated d-dimer and low fibrinogen (70 mg/dL). SRA was strongly positive, a diagnosis of delayed-onset HIT was made, and he was treated with therapeutic plasma exchange (TPE). Mass-Fix was negative for M-proteins (FIG. 7A). High-resolution mass spectrometry analysis demonstrated striking mass similarities between 3 of the patient's overexpressed IgG lambda micro M-proteins and the patients anti-PF4 antibody (upper tables) suggesting that the anti-PF4 antibody was an oligoclonal antibody (FIG. 7B). FIG. 7C and FIG. 7D demonstrate that the PF4-heparin bead eluate, as opposed to control beads (PBS-treated heparin beads) has platelet activating activity and binding to PF4-PVS, respectively, demonstrating that the PF4 heparin bead eluate (which has the monoclonal antibody) has anti-PF4 antibody activity. SRA activity in the PF4 and control Heparin Sepharose® bead eluates, similarly, were at 84% vs 0%, respectively.

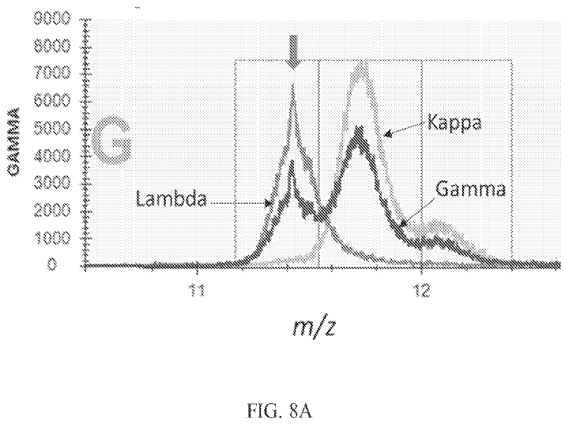

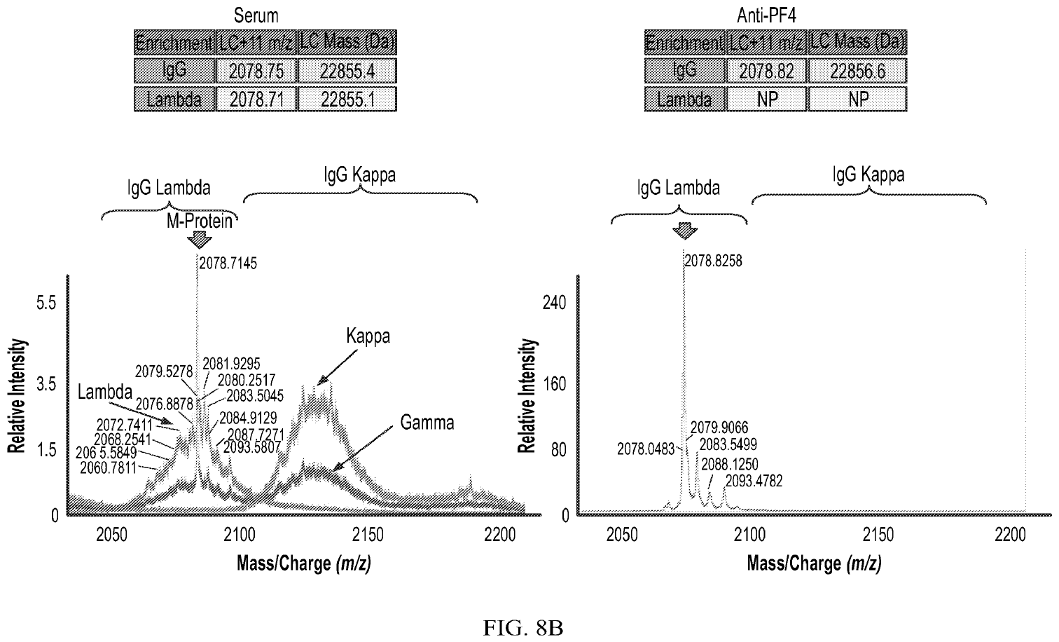

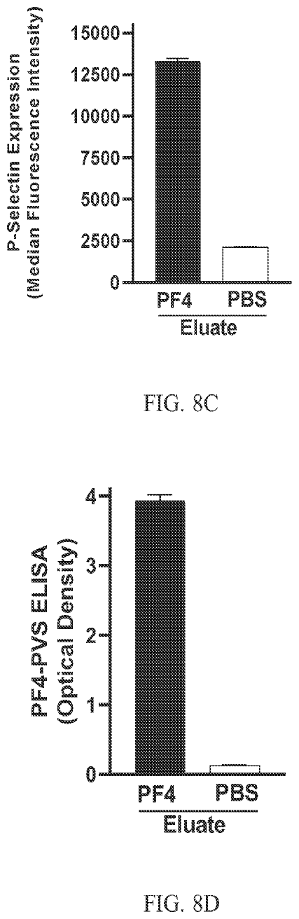

Patient 5 (PT5) was a 74-year-old male who underwent coronary artery bypass surgery and was noted to have shortness of breath due to pulmonary embolus three days later. In addition, perfusion of his lower extremity was noted to be compromised which progressed to cyanosis and gangrene. Platelet count was noted to be 100,000/μL and heparin-dependent platelet aggregation testing was positive. The disease progressed despite TPE therapy and the patient died due to hepatic and renal failure. Review of Mass-Fix results revealed the presence of an IgG lambda M-protein (FIG. 8A). High-resolution mass spectrometry analysis demonstrated striking mass similarities between the patients overexpressed IgG lambda M-protein and the patients anti-PF4 antibody (upper tables) suggesting that the anti-PF4 antibody is a monoclonal antibody (FIG. 8B). FIG. 8C and FIG. 8D demonstrate that the PF4-heparin bead eluate, as opposed to control beads (PBS-treated heparin beads) has platelet activating activity and binding to PF4-PVS, respectively, demonstrating that the PF4 heparin bead eluate (which has the monoclonal antibody) has anti-PF4 antibody activity. SRA activity in the PF4 and control Heparin Sepharose® bead eluates, similarly, were at 78% vs 3%, respectively.

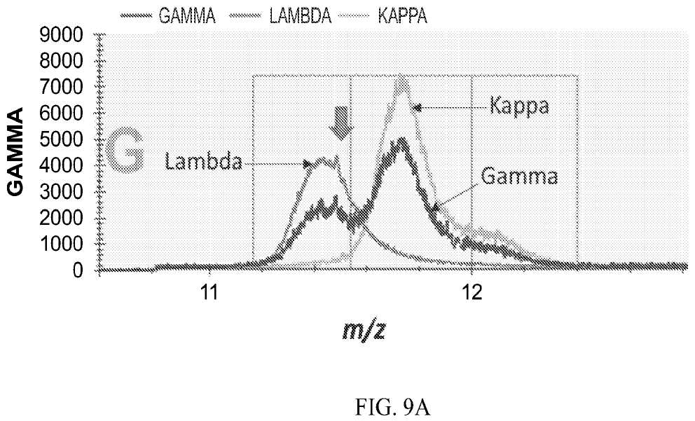

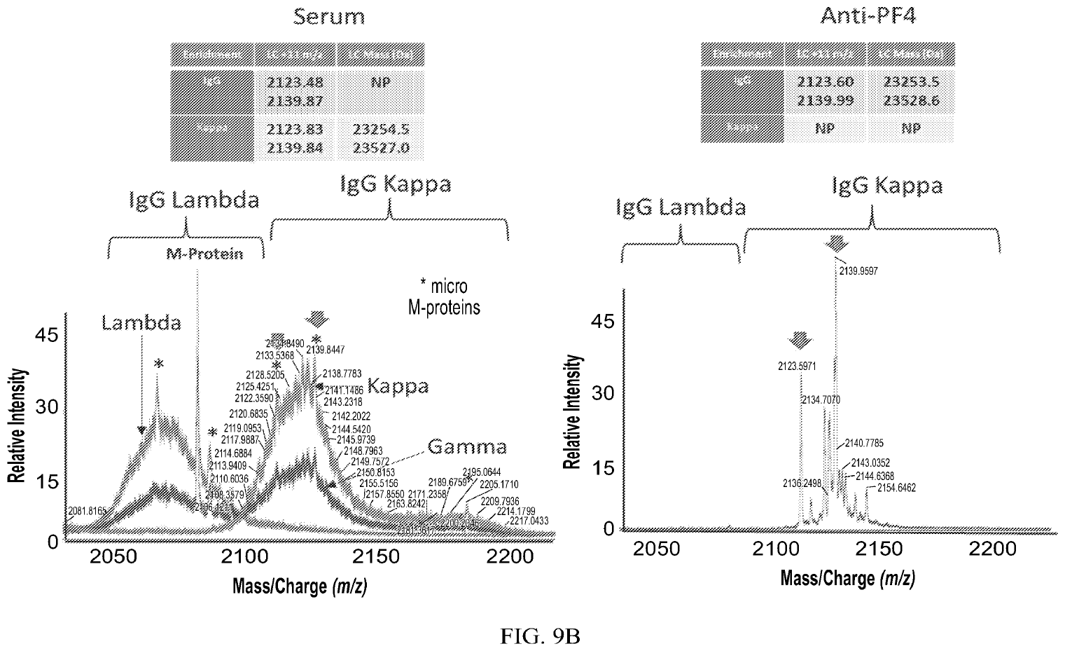

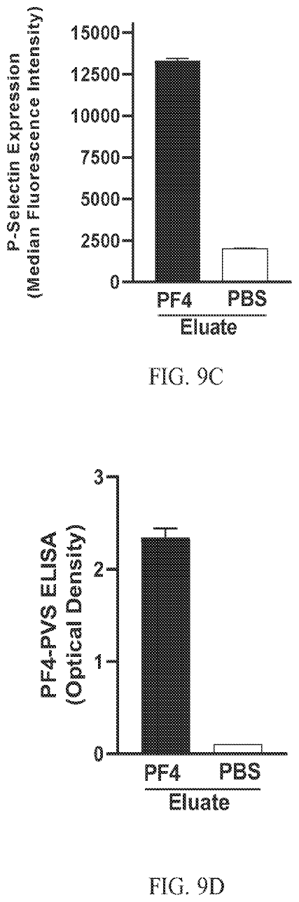

Patient 6 (PT6) is a 64-year-old female who developed HIT after intraoperative and post-operative heparin therapy and was complicated by DVT, limb gangrene and renal failure. SRA was reported as strongly positive and the patient was treated with danaparoid and TPE. Review of Mass-Fix results revealed the presence of a small IgG lambda M-protein (FIG. 9A). High-resolution mass spectrometry analysis demonstrated that mass similarities between the patients overexpressed IgG kappa micro M-proteins and the patients anti-PF4 antibody (upper tables) suggesting that the anti-PF4 antibody is oligoclonal; the IgG lambda M protein was unrelated to anti-PF4 antibodies (FIG. 9B). FIG. 9C and FIG. 9D demonstrate that the PF4-heparin bead eluate, as opposed to control beads (PBS-treated heparin beads) has platelet activating activity and binding to PF4-PVS, respectively, demonstrating that the PF4 heparin bead eluate (which has the oligoclonal antibodies) has anti-PF4 antibody activity. SRA activity in the PF4 and control Heparin Sepharose® bead eluates, similarly, were at 93% vs 2%, respectively.

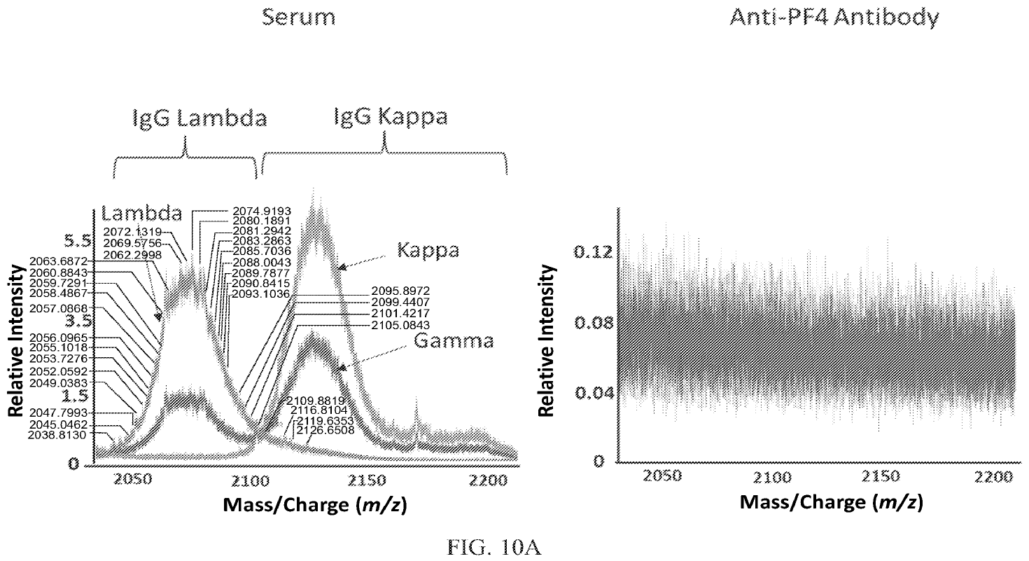

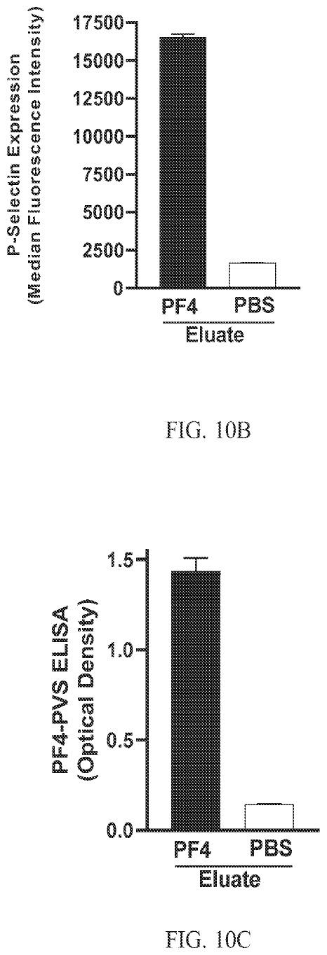

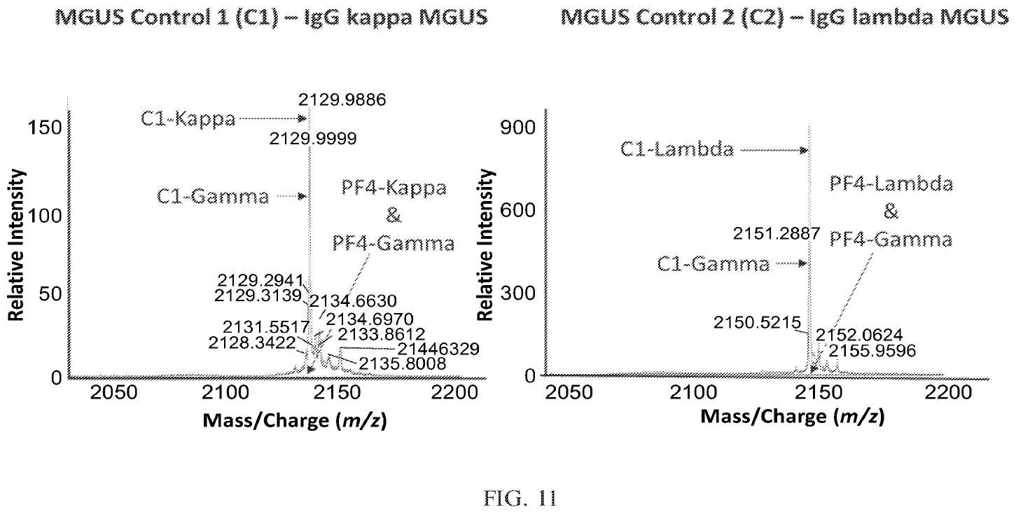

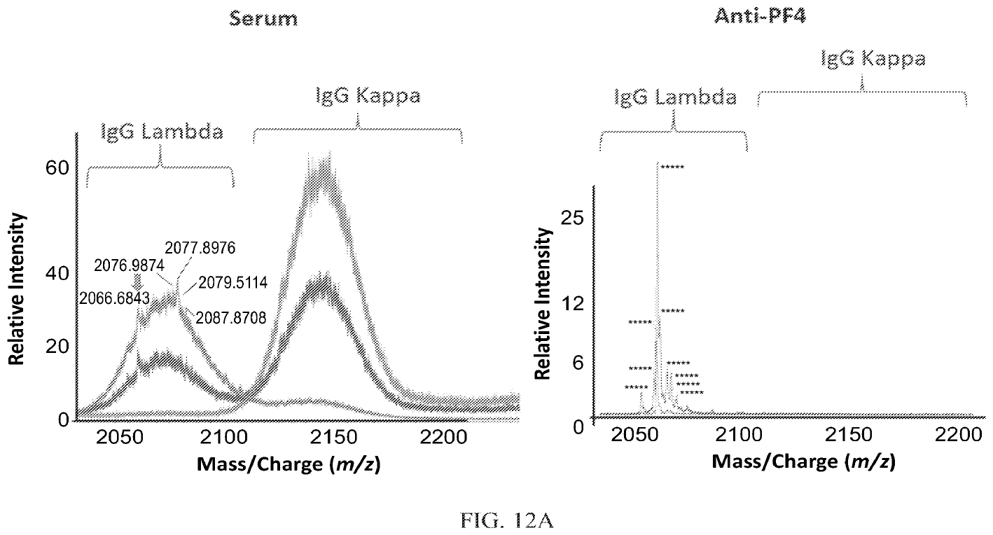

Patient 7 (PT7) was a 37-year-old female with no known past medical history, who was admitted for shortness of breath due to covid-19 infection but clinically worsened and required ventilator support. Her course was complicated by sepsis and HIT (ELISA-positive: SRA modestly positive at 62%). The patient was included in the analysis as a control case of relatively “mild” HIT. No monoclonal/oligoclonal antibodies were noted in the PF4-Heparin Sepharose® bead eluates (FIG. 10A). FIG. 10B and FIG. 10C demonstrate that the PF4-heparin bead eluate, as opposed to control beads (PBS-treated heparin beads) had platelet activating activity and binding to PF4-PVS, respectively, demonstrating that the PF4 heparin bead eluate has anti-PF4 antibody activity. SRA activity in the PF4 and control Heparin Sepharose® bead eluates, similarly, were at 93% vs 10%, respectively.

Serum from two MGUS patient controls without thrombosis/thrombocytopenia-suspected anti-PF4 antibodies were subject to processing with PF4-Heparin SepharoseR beads as for the prior patients to evaluate non-specific binding of M-proteins to PF4-Heparin Sepharose® beads. MGUS-C1 had an IgG kappa M-protein while MGUS-C2 had an IgG lambda M-protein (FIG. 11). Results demonstrated that no significant non-specific binding of the M-protein to the PF4 enrichment beads was noted (FIG. 11) for either MGUS-C1 or MGUS-C2.

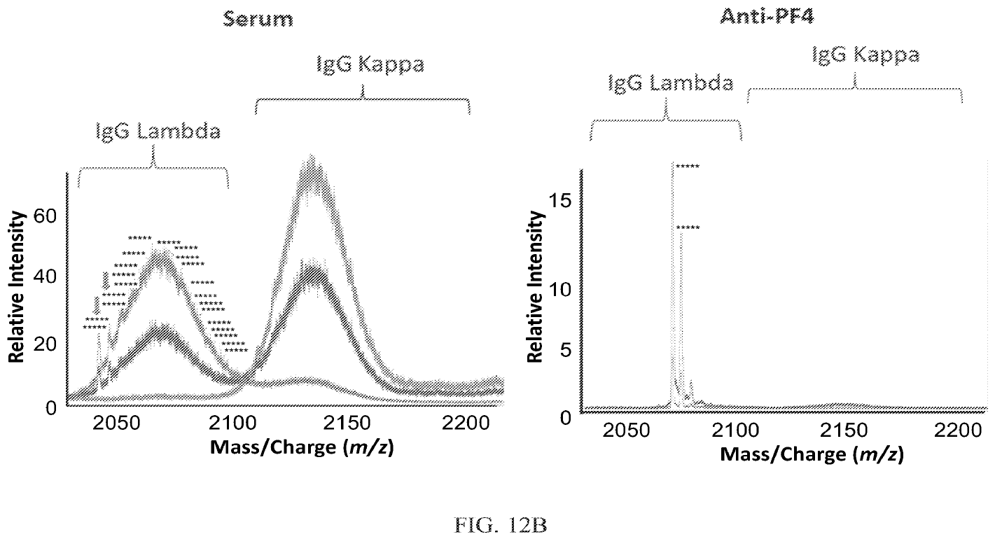

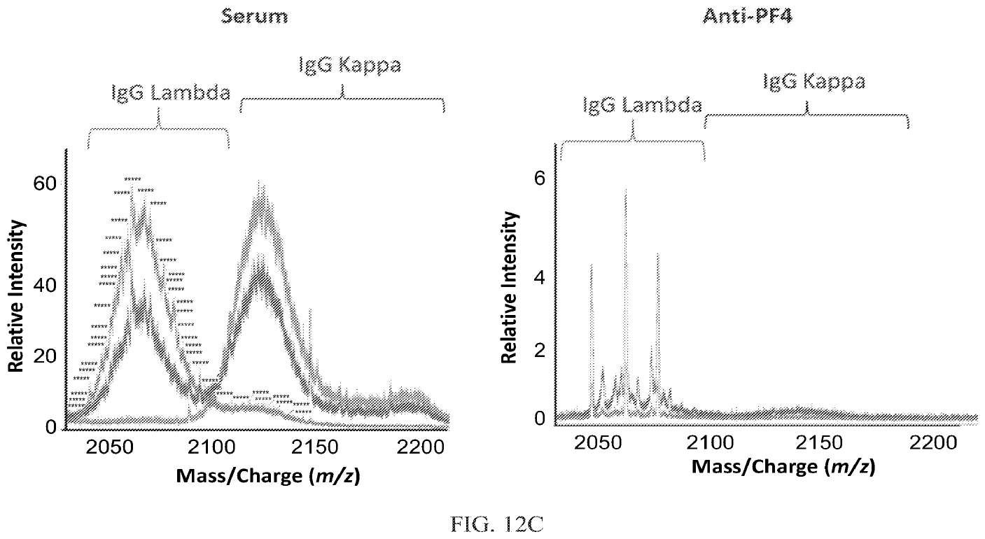

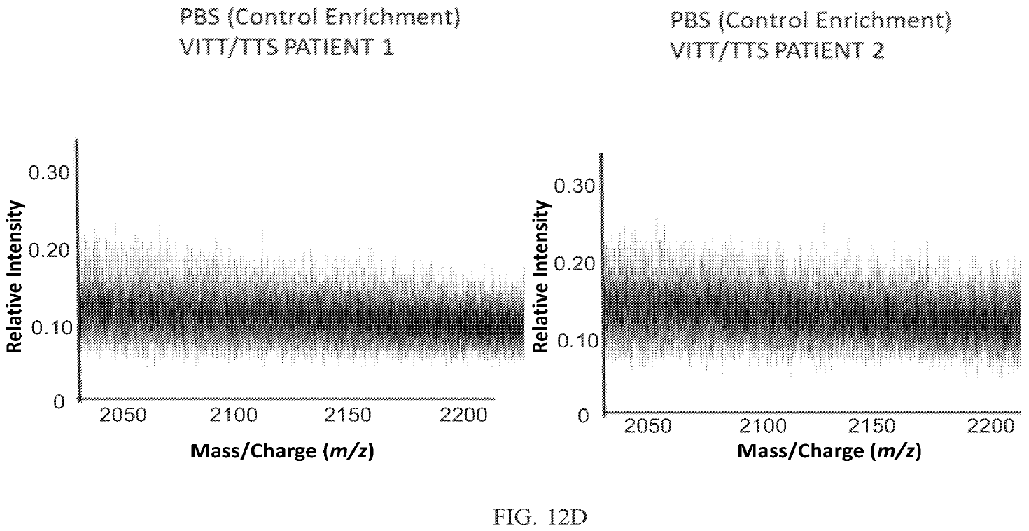

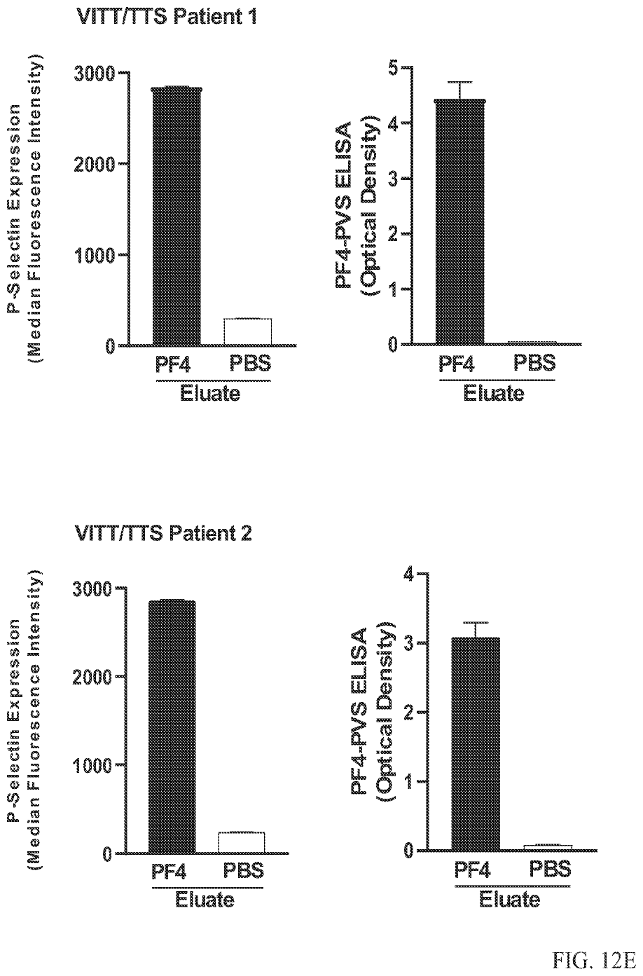

VITT/TTS Patients 1, 2 and 3 were individuals who developed thrombocytopenia and thrombosis after covid-19 vaccination due to development of anti-PF4 antibodies. High-resolution mass spectrometry analysis of the serum of these three patients demonstrated multiple micro M-proteins. One, two and three micro M-proteins demonstrated striking mass similarities with the patient's anti-PF4 antibody(ies) suggesting that the anti-PF4 antibody was a monoclonal (FIG. 12A), biclonal (FIG. 12B) or triclonal (FIG. 12C), respectively. Control enrichment of HIT antibodies from VITT/TTS patients 1 and 2 did not show any antibodies (FIG. 12D; Inadequate sample to perform testing on VITT/TTS patient 3). Only PF4-heparin purified eluates demonstrated platelet-activation and binding to PF4-PVS complexes in ELISA (FIG. 12E) as opposed to control Heparin Sepharose® beads demonstrating that the PF4 heparin bead eluate (which has the M-protein) had anti-PF4 antibody activity.

### Example 2: Treating MGTS

A sample (e.g., serum or plasma) is obtained from a human having one or more thrombotic and/or thrombocytopenia conditions. The sample is assessed for the presence or absence of a population of monoclonal antibodies having binding specificity for a PF4 polypeptide. In some cases, a mass spectrometry technique (e.g., ESI-Q-TOF mass spectrometry) is performed to detect the presence or absence of a population of monoclonal antibodies having binding specificity for a PF4 polypeptide. When the presence of a population of monoclonal antibodies having binding specificity for a PF4 polypeptide is detected in the sample, then the human is identified as having, or as being likely to develop, MGTS, and is administered one or more MGTS treatments (e.g., a B-cell targeted therapy, a plasma cell targeted therapy, plasmapheresis, therapeutic plasma exchange, and administering intravenous immunoglobulin G).

### Example 3: Treating Thrombotic and or Thrombocytopenia Conditions

A sample (e.g., serum or plasma) is obtained from a human having one or more thrombotic and/or thrombocytopenia conditions. The sample is assessed for the presence or absence of a population of monoclonal antibodies having binding specificity for a PF4 polypeptide. In some cases, a mass spectrometry technique (e.g., ESI-Q-TOF mass spectrometry) is performed to detect the presence or absence of a population of monoclonal antibodies having binding specificity for a PF4 polypeptide. When the absence of a population of monoclonal antibodies having binding specificity for a PF4 polypeptide is detected in the sample, then the human is identified as not having, or as not being likely to develop, MGTS, and is administered one or more treatments for thrombotic and/or thrombocytopenia conditions (e.g., administering an anticoagulant, administering a thrombolytic, therapeutic plasma exchange, and administering intravenous immunoglobulin G).

### Example 4: Treating MGTS

A sample (e.g., serum or plasma) is obtained from a human having one or more thrombotic and/or thrombocytopenia conditions. The sample is assessed for the presence or absence of a population of oligoclonal antibodies having binding specificity for a PF4 polypeptide. In some cases, a mass spectrometry technique (e.g., ESI-Q-TOF mass spectrometry) is performed to detect the presence or absence of a population of oligoclonal antibodies having binding specificity for a PF4 polypeptide. When the presence of a population of oligoclonal antibodies having binding specificity for a PF4 polypeptide is detected in the sample, then the human is identified as having, or as being likely to develop, MGTS, and is administered one or more MGTS treatments (e.g., a B-cell targeted therapy, a plasma cell targeted therapy, plasmapheresis, therapeutic plasma exchange, and administering intravenous immunoglobulin G).

### Example 5: Treating Thrombotic and or Thrombocytopenia Conditions

A sample (e.g., serum or plasma) is obtained from a human having one or more thrombotic and/or thrombocytopenia conditions. The sample is assessed for the presence or absence of a population of oligoclonal antibodies having binding specificity for a PF4 polypeptide. In some cases, a mass spectrometry technique (e.g., ESI-Q-TOF mass spectrometry) is performed to detect the presence or absence of a population of oligoclonal antibodies having binding specificity for a PF4 polypeptide. When the absence of a population of oligoclonal antibodies having binding specificity for a PF4 polypeptide is detected in the sample, then the human is identified as not having, or as not being likely to develop, MGTS, and is administered one or more treatments for thrombotic and/or thrombocytopenia conditions (e.g., administering an anticoagulant, administering a thrombolytic, therapeutic plasma exchange, and administering intravenous immunoglobulin G).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting a population of monoclonal antibodies specific for a platelet factor 4 (PF4) polypeptide in a sample obtained from a mammal, said method comprising:
immunopurifying antibodies specific for said PF4 polypeptide from the sample;
subjecting the immunopurified immunoglobulins to a mass spectrometry technique to obtain a mass spectrum of the sample; and
identifying the presence of said population of monoclonal antibodies specific for said PF4 polypeptide based on a peak in the spectrum corresponding to the monoclonal antibodies specific for said PF4 polypeptide.

2. A method for detecting a population of oligoclonal antibodies specific for a platelet factor 4 (PF4) polypeptide in a sample obtained from a mammal, said method comprising:
immunopurifying antibodies specific for said PF4 polypeptide from the sample;
subjecting the immunopurified immunoglobulins to a mass spectrometry technique to obtain a mass spectrum of the sample; and
identifying the presence of said population of oligoclonal antibodies specific for said PF4 polypeptide based on a peak in the spectrum corresponding to the monoclonal antibodies specific for said PF4 polypeptide.

3-5. (canceled)

6. The method of claim 1, wherein said sample is a serum sample or a plasma sample.

7. The method of claim 1, wherein said mammal is a human.

8-10. (canceled)

11. A method for assessing a mammal having a thrombotic and/or thrombocytopenia condition, wherein said method comprises:
(a) detecting, in a sample from said mammal, a presence or absence of a population of monoclonal antibodies having binding specificity for a PF4 polypeptide;
(b) classifying said mammal as having monoclonal gammopathy of thrombotic/thrombocytopenia significance (MGTS) if said presence of said population is detected; and
(c) classifying said mammal as not having MGTS if said absence of said population level is detected.

12. (canceled)

13. The method of claim 11, wherein said thrombotic and/or thrombocytopenia condition is selected from the group consisting of heparin-induced thrombocytopenia (HIT), HIT with thrombosis, delayed-onset HIT, flush-related HIT, persistent HIT, HIT with disseminated intravascular coagulation, refractory HIT, spontaneous HIT with thrombosis, spontaneous HIT without thrombosis, and vaccine-induced immune thrombotic thrombocytopenia (VITT).

14-16. (canceled)

17. The method of claim 11, wherein said method comprises, prior to said detecting, immunopurifying said sample for antibodies specific for said PF4 polypeptide.

18. (canceled)

19. The method of claim 11, wherein said sample is a serum sample or a plasma sample.

20. The method of claim 11, wherein said mammal is a human.

21-23. (canceled)

24. A method for treating a mammal having a thrombotic and/or thrombocytopenia condition, wherein said method comprises:
(a) detecting, in a sample obtained from said mammal, a population of monoclonal antibodies having binding specificity for a PF4 polypeptide; and
(b) administering a therapy effective to treat a monoclonal gammopathy to said mammal.

25. (canceled)

26. The method of claim 24, wherein said therapy is selected from the group consisting of a B-cell targeted therapy, a plasma cell targeted therapy, plasmapheresis, therapeutic plasma exchange, and administering intravenous immunoglobulin G.

27-29. (canceled)

30. The method of claim 24, wherein said method comprises, prior to said detecting, immunopurifying said sample for antibodies specific for said PF4 polypeptide.

31. (canceled)

32. The method of claim 24, wherein said sample is a serum sample or a plasma sample.

33. The method of claim 24, wherein said mammal is a human.

34-36. (canceled)

37. A method for treating a mammal having a thrombotic and/or thrombocytopenia condition, wherein said method comprises:
(a) detecting, in a sample obtained from said mammal, an absence of a population of monoclonal antibodies having binding specificity for a PF4 polypeptide; and
(b) administering a therapy effective to treat thrombosis to said mammal.

38. (canceled)

39. The method of claim 37, wherein said therapy is selected from the group consisting of administering an anticoagulant, administering a thrombolytic, therapeutic plasma exchange, and administering intravenous immunoglobulin G.

40-42. (canceled)

43. The method of claim 38, wherein said method comprises, prior to said detecting, immunopurifying said sample for antibodies specific for said PF4 polypeptide.

44. (canceled)

45. The method of claim 38, wherein said sample is a serum sample or a plasma sample.

46. The method of claim 38, wherein said mammal is a human.
